(19) United States
(12) Patent Application Publication
(10) Pub. No.: US 2012/0219545 A1
(43) Pub. Date: Aug. 30, 2012

(54) MATERIALS AND METHODS FOR DIAGNOSING AND TREATING SHELLFISH ALLERGY

(75) Inventors: Rosalia Ayuso, Middle Village, NY (US); Hugh A. Sampson, Greenwich, CT (US); Galina Grishina, Tarry Town, NY (US)

(73) Assignee: MOUNT SINAI SCHOOL OF MEDICINE, New York, NY (US)

(21) Appl. No.: 13/387,894

(22) PCT Filed: Aug. 2, 2010
(86) PCT No.: PCT/US10/44084

§ 371 Date: May 14, 2012

Related U.S. Application Data
(60) Provisional application No. 61/230,482, filed on Jul. 31, 2009.

(51) Int. Cl. A61K 39/395; A61P 37/08; G01N 33/53

(52) U.S. Cl. 424/133.1; 424/172.1; 424/152.1; 436/501; 435/7.92; 424/139.1

(57) ABSTRACT

The disclosure provides materials and methods for diagnosing, treating and preventing shellfish allergic reactions, including allergic reactions to shrimp. The technology involves one or more shellfish-specific proteins selected from the group of myosin light chain, sarcoplasmic calcium-binding protein, hemocyanin, fatty acid binding protein, and troponin C, for example from shrimp, as well as encoding polynucleotides, vectors host cells, and specific binding partners for such proteins, e.g., antibodies. In compositions comprising a plurality of shellfish allergens, any of the aforementioned proteins may be included, as may arginine kinase and tropomyosin. The methods according to the disclosure include methods of making the specific binding partners such as antibodies as well as methods of using the materials of the disclosure to diagnose, treat or prevent an allergic reaction to shellfish, e.g., shrimp.

### RELATED APPLICATIONS

This application claims the benefit of priority of U.S. Provisional Patent Application Ser. No. 61/230,482, filed Jul. 31, 2009, which is incorporated herein by reference in its entirety.

### FIELD OF INVENTION

The present invention relates to materials and methods for diagnosing, preventing or treating allergic reactions to shellfish and, in particular, to allergic reactions to shrimp.

### BACKGROUND

Allergic reactions to shellfish, e.g., shrimp, range from mild to systemic reactions, including severe anaphylactic reactions. Beyond allergic reactions to consumption of recognizable shellfish, shellfish products can make their way into a variety of foodstuffs that effectively mask the dangers. As the number of people worldwide exhibiting symptoms of shellfish allergy remains at significant levels, the problem of allergic reactions to shellfish continues to pose a significant health problem.

Shellfish allergy is a long-lasting and potentially life-threatening disorder. Most shellfish species provoking allergic reactions belong to the class Crustacea, which includes shrimp, prawn, crab, lobster, and crawfish. A recent survey found that one in fifty Americans had shellfish allergy. Shellfish are the number one cause of food allergy in adults in the United States and are responsible for the majority of emergency department visits for food allergy, not only in adults but also in children 6 years of age and older, and a significant cause of allergic reactions in children one to five years old.

A large variety of crustaceans are consumed by humans. Although the black tiger shrimp (Penaeus monodon) is the most widely cultured prawn species in the world, the Pacific white shrimp (Litopenaeus vannamei), which is actually a prawn, is the species of choice in the shrimp farming industry in the Western hemisphere. Together these two species account for 80% of all farmed shrimp. Shrimp consumption has more than tripled since 1970, and it is expected that allergy to shellfish will continue to increase. Therefore, a better understanding is needed of shrimp proteins involved in the development of allergic reactions.

Until recently, the muscle protein tropomyosin was the only major cross-reactive allergen identified in different shrimp species. Shrimp tropomyosin has been shown to inhibit 80% of patients' IgE RAST (radioallergosorbent) reactivity to whole-body shrimp extract, indicating that tropomyosin is responsible for most of the allergenic activity of shrimp.

In spite of the high prevalence of shellfish allergy, few options are available for treatment, and avoidance is the only therapy recommended. However, the frequency and severity of reactions after accidental exposure to shellfish make it necessary to develop improved diagnostic and therapeutic options for shellfish allergy.

Thus, there remains a need in the art to provide materials and methods to diagnose, treat and/or prevent allergic reactions to shellfish such as shrimp.

### SUMMARY

The technology disclosed herein satisfies at least one of the aforementioned needs in the art in providing shellfish (e.g., shrimp) allergens as well as specific binding partners of such allergens, e.g., antibodies, and methods for generating such specific binding partners, methods of diagnosing the potential for an allergic reaction, methods for treating an allergic reaction, and methods for preventing an allergic reaction, such as by subjecting an individual at risk of an allergic reaction to vaccinating levels of at least one of the allergens.

In one aspect, the disclosure provides a method of treating a shellfish allergic reaction comprising administering a therapeutically effective amount of a specific binding partner of a shellfish protein selected from the group consisting of myosin light chain, sarcoplasmic calcium-binding protein, fatty acid binding protein (FABP), hemocyanin and troponin C. In some embodiments, the shellfish protein is derived from shrimp, prawn, crab, lobster or crawfish. In embodiments in which the shellfish protein is derived from a shrimp, any shrimp source may be used, such as a shrimp selected from the group consisting of black tiger shrimp and white shrimp. This aspect of the disclosure comprehends embodiments in which the specific binding partner is an antibody, such as a monoclonal antibody, or antibody fragment.

A related aspect according to the disclosure provides a method as described above further comprising administering a therapeutically effective amount of a second binding partner specifically recognizing a second shellfish protein selected from the group consisting of myosin light chain, sarcoplasmic calcium binding protein, tropomyosin, arginine kinase, hemocyanin, fatty acid binding protein and troponin C, wherein the first and second shellfish proteins are different. In some embodiments, the second shellfish protein is derived from shrimp, prawn, crab, lobster or crawfish. In embodiments in which the second shellfish protein is derived from a shrimp, any shrimp source may be used, such as a shrimp selected from the group consisting of black tiger shrimp and white shrimp. In some embodiments of the method, the specific binding partner is an antibody, such as a monoclonal antibody, or antibody fragment.

Another aspect according to the disclosure is a method of ameliorating or preventing a shellfish allergic reaction comprising administering a therapeutically effective amount of a protein selected from the group consisting of shellfish myosin light chain, shellfish sarcoplasmic calcium-binding protein, fatty acid binding protein (FABP), hemocyanin, troponin C and a specific binding partner thereof. In some embodiments, the shellfish protein is derived from shrimp, prawn, crab, lobster or crawfish. In embodiments in which the shellfish protein is derived from a shrimp, any shrimp source may be used, such as a shrimp selected from the group consisting of black tiger shrimp and white shrimp. In some embodiments, the specific binding partner is an antibody, such as a monoclonal antibody, or antibody fragment.

In a related aspect, the disclosure provides a method as described immediately above, further comprising administering a therapeutically effective amount of a second protein selected from the group consisting of shellfish myosin light chain, shellfish sarcoplasmic calcium-binding protein, shellfish tropomyosin, shellfish arginine kinase, shellfish hemocyanin, shellfish fatty acid binding protein, shellfish troponin C and a specific binding partner thereof, wherein the first and second proteins are different. In some embodiments, the second shellfish protein is derived from shrimp, prawn, crab, lobster or crawfish. In embodiments in which the second shellfish protein is derived from a shrimp, any shrimp source may be used, such as a shrimp selected from the group consisting of black tiger shrimp and white shrimp. In some embodiments, the specific binding partner is an antibody, such as a monoclonal antibody, or antibody fragment.

Yet another aspect according to the disclosure provides a method of diagnosing a risk of an allergic reaction to shellfish comprising contacting an immunoglobulin-containing biological sample of a subject with a shellfish protein selected from the group consisting of myosin light chain, sarcoplasmic calcium binding protein, hemocyanin, fatty acid binding protein and troponin C, and measuring a reaction between the sample and the protein, wherein a reaction leads to a diagnosis of a subject at risk of a shellfish allergic reaction. In some embodiments, the shellfish protein is derived from shrimp, prawn, crab, lobster or crawfish. In embodiments in which the shellfish protein is derived from a shrimp, any shrimp source may be used, such as a shrimp selected from the group consisting of black tiger shrimp and white shrimp.

In a related aspect, the disclosure provides a method of diagnosis as described immediately above, further comprising contacting the biological sample with a second shellfish protein selected from the group consisting of myosin light chain, sarcoplasmic calcium binding protein, tropomyosin, arginine kinase, hemocyanin, fatty acid binding protein and troponin C, wherein the first and second shellfish proteins are not the same protein. In some embodiments, the second shellfish protein is derived from shrimp, prawn, crab, lobster or crawfish. In embodiments in which the second shellfish protein is derived from a shrimp, any shrimp source may be used, such as a shrimp selected from the group consisting of black tiger shrimp and white shrimp.

In a further aspect, the disclosure provides methods of treating, ameliorating or preventing a shellfish allergic reaction comprising administering a therapeutically effective amount of a protein selected from the group consisting of shellfish myosin light chain, shellfish sarcoplasmic calcium-binding protein, shellfish fatty acid binding protein, shellfish hemocyanin, shellfish troponin C and a monoclonal antibody or fragment thereof. In more particular aspects, the monoclonal antibody or fragment thereof binds to an epitope comprising an amino acid at any one of amino acid positions 1-18, 25-42, 64-96, 121-141, 142-159, 160-192, 232-255, or 319-342 in the amino acid sequence of SEQ ID NO: 58; an amino acid at any one of amino acid positions 13-30, 22-48, 49-66, 58-90, 79-99, or 118-141 in the amino acid sequence of SEQ ID NO: 59; an amino acid at any one of amino acid positions 10-36, 49-72, or 130-147 in the amino acid sequence of SEQ ID NO: 60; or an amino acid at any one of amino acid positions 1-36, 37-63, 61-81, 82-105, 115-150, 142-162, 157-183, 190-210, or 246-284 in the amino acid sequence of SEQ ID NO: 61. In certain aspects, the monoclonal antibody is a recombinant antibody. In further aspects, the recombinant antibody is selected from the group consisting of a human chimeric antibody, a humanized antibody, and a human antibody.

The invention includes uses of compositions of the invention, including binding partners, antibodies and fragments thereof, and proteins, for the preparation of medicaments. Other related aspects are also provided in the instant invention.

The foregoing summary is not intended to define every aspect of the invention, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the invention, are given by way of illustration only, because various changes and modifications within the spirit and scope of the invention will become apparent to those skilled in the art from this detailed description.

### BRIEF DESCRIPTION OF THE DRAWING

FIG. 1 shows IgE immunoblotting of 38 patients' sera to boiled (BS, top) and raw (RS, bottom) shrimp extract. M, molecular weight; P, total shrimp protein; lanes 1 to 38, immunolabeling with patients' sera; NC, negative control. IgE reactivity to tropomyosin is represented at 34 kd and IgE reactivity to MLC is represented at 20 kd.

FIG. 2 provides a 2-D proteomics map and immunolabeling of boiled shrimp extract. Left, Gel stained for total protein analysis; right, IgE immunolabeling with one representative subject. The circle shows the spot used for MALDI/MS analysis. The square shows two amino acid sequences obtained by means of Edman sequencing, i.e., KGGXNVFDMFTQK (SEQ ID NO: 1) and SSGESDDDDVVAASIR (SEQ ID NO: 2). MW, Molecular weight; p1, isoelectric point.

FIG. 3 discloses immunoblot inhibition of IgE reactivity to boiled shrimp extract with recombinant tropomyosin. M, molecular weight; P, protein staining of boiled shrimp extract; lane 3, IgE immunoblotting with a serum pool; lane 4, pool preincubated with recombinant tropomyosin (Inh Tp); and lane 5, pool preincubated with chickpea extract as a control (Inh C).

FIG. 4 shows IgE binding to recombinant shrimp MLC. Numbered lanes, immunoblotting with patients' sera. NC, negative control; Prot, amido black staining of recombinant MLC (arrow).

FIG. 5 shows an IgE immunoblot of a representative group of 14 patients to BS (boiled shrimp) extract (A) and recombinant sarcoplasmic calcium-binding protein (rSCP) (B). P, protein staining of total shrimp protein (A), rSCP (B). Lanes, Immunolabeling with 14 patients' sera; NC, negative controls (two used in A, three in B). The sera of patients 2 and 43 were used for ELISA experiments described herein; sera of patients 14, 47 and 48 were used in RBL assays, also described herein. Levels of shrimp-specific IgE are noted in kU_{A}/L.

FIG. 6 provides a multiple sequence alignment of protein sequences of SCPs. Sequence identities of Lit v 4.0101 with Penaeus spp α chain (P02636; 93.8%), β chain (P02635, 80%), crayfish SCP (P. leptodactylus; ABB58783, P05946; 81% to 82%), scallop (M. yessoensis), SCP (P02637; 14%), and fruit fly SCP (D. melanogaster, NP_{—}001015389 and NP_{—}524381; 18% to 52%). Identical amino acids to L. vannamei SCP are replaced by dashes. Boxed peptide regions of SCP α-B and A chains—Penaeus—P02636 were identified by MS/MS as matching the amino acid sequence of SCP a chain; boxed peptide regions of SCP β chain—Penaeus—P02635 were identified by MS/MS as matching the amino acid sequence of SCP β chain.

FIG. 7 discloses immunoblot inhibition of IgE reactivity to rSCP with extracts from other arthropods and mollusks. P, Protein staining of rSCP. Lane 1, pool without inhibitor. Lanes 2-9, pool preincubated with extract of D. farinae (2), B. germanica (3), boiled lobster (4), crab (5), squid (6), scallop (7), mussel (8), shrimp (9), and chickpea (10) as negative control.

FIG. 8 provides graphs showing ELISA inhibition and mediator release from an RBL cell line. ELISA inhibition of BS with serum from subject 2 (A) and subject 43(B). Inhibitors: rSCP, rLit v 1, BS, and rCor a 9. Inhibitor concentrations ranged from 25×10⁻⁸ μg/ml (shown as 10⁻⁸) to 25 μg/ml total protein. β-hexosaminidase release from RBL cells induced by rSCP, rLit v 1, and BS (maximal concentration, 1 μg/ml) is expressed as a percentage of total possible release induced by Triton X-100. Cells were sensitized with serum pool of subjects 14, 47 and 48 (C).

### DETAILED DESCRIPTION

Allergic reactions to shellfish (e.g., shrimp) range from mild to systemic reactions, including severe anaphylactic reactions. In accordance with the disclosure, several shellfish allergens have been identified that can be utilized in the diagnosis and treatment of patients with shellfish (e.g., shrimp) allergy. In addition to the identification of sarcoplasmic calcium-binding protein as a shellfish (e.g., shrimp) allergen, Myosin light chain (MLC) is identified as a new major allergenic shrimp protein, particularly in children. Initial studies revealed that IgE recognition of MLC in boiled shrimp (BS) extract was very intense, but the recombinant MLC protein was significantly less recognized. Although this may be a result of posttranslational modifications present in the native form, the possibility of another IgE-binding protein of similar molecular weight was considered. With additional investigation, the proteins identified as shellfish (e.g., shrimp) allergens are myosin light chain (MLC; SEQ ID NO: 59), sarcoplasmic calcium-binding protein (SCP; SEQ ID NO: 60), hemocyanin (SEQ ID NOs: 56 and 57), fatty acid binding protein (FABP; SEQ ID NOs: 7 and 8) and troponin C (SEQ ID NOs: 17 and 18). The World Health Organization/International Union of Immunological Societies Allergen Nomenclature Subcommittee has designated shrimp tropomyosin as Lit v 1, shrimp arginine kinase as Lit v 2, shrimp MLC as Lit v 3 and shrimp SCP as Lit v 4. The proteins, polypeptides and peptides of Lit v 3 (shrimp myosin light chain), Lit v 4 (shrimp sarcoplasmic calcium-binding protein), shrimp hemocyanin, shrimp FABP and shrimp troponin C are recognized by serum Immunoglobulin E (IgE) from patients with shrimp allergy.

The disclosure provides proteins, polypeptides and peptides comprising at least one epitope of MLC, SCP, FABP, hemocyanin or troponin C. Isolated and purified proteins, polypeptides and peptides may be made by introducing a nucleic acid encoding the protein, polypeptide or peptide into a suitable host cell, for example by transformation, transfection or injection, culturing the host cell under conditions suitable for expression, and recovering the recombinant polypeptide or peptide. The recombinant product may be recovered from cells or culture medium by methods known in the art. The polypeptides and peptides may also be made by well known methods of protein synthesis, such as solid-phase peptide synthesis.

Biologically active analogs of the polypeptides and peptides are similarly made utilizing a nucleic acid encoding a biologically active analog. A biologically active analog is one which maintains the ability to be recognized by serum from patients having a shellfish allergy. The term “analogs” includes substitutions and alterations of the amino acid sequences described herein, provided that the substitutions and alterations do not eliminate biological activity. Amino acid insertional derivatives include amino- and carboxy-terminal fusions and single or multiple intra-sequence insertions. Deletional variants have one or more amino acids removed from the protein, polypeptide or peptide. In substitutional amino acid variants, at least one residue has been replaced by a different residue. Biologically active analogs may be made by recombinant methods, e.g., as described in Sambrook et al. or by peptide synthetic techniques well-known in the art, such as solid-phase peptide synthesis. Fusion proteins comprising the polypeptides and peptides of the disclosure are also provided. The protein, polypeptide or peptide may be fused, for example, to P-galactosidase or glutathione-S-transferase, or to any other protein, e.g., to facilitate processing, purification or immobilization.

The disclosure also provides isolated nucleic acids encoding the allergenic proteins, polypeptides and peptides described herein, i.e., nucleic acids that encode Lit v 3 (shrimp myosin light chain), Lit v 4 (shrimp sarcoplasmic calcium-binding protein), shrimp hemocyanin, and/or shrimp FABP, each of which is recognized by an antibody specific for a shrimp allergen. Exemplary polynucleotide coding sequences and the protein gene product sequences encoded thereby are provided for shrimp myosin light chain (Genbank Acc. No. EU449515; SEQ ID NOs: 3 and 4), sarcoplasmic calcium binding protein (Genbank Acc. No. FJ184279; SEQ ID NOs: 5 and 6), FABP (Genbank Acc. No. DQ459988; SEQ ID NOs: 7 and 8), hemocyanin subunit L(Genbank Acc. No. EF375711; SEQ ID NOs: 9 and 10), hemocyanin (SEQ ID NOS: 56 and 57), including hemocyanin subunit Y (Genbank Acc. No. EF375712; SEQ ID NOs: 11 and 12), tropomyosin (Genbank Acc. No. EU410072; SEQ ID NOs: 15 and 16), and troponin C (Genbank Acc. No. BC071546; SEQ ID NOs: 17 and 18).

The disclosure provides isolated nucleic acids that encode a protein, polypeptide or peptide comprising at least one epitope. IgE epitopes of each of the above allergens may be identified by providing a shrimp allergen epitope library and screening the library against serum from allergic patients. Methods for creating and screening epitope libraries are known in the art and are disclosed, for example, by Scott et al., Science 249: 386 (1990). Epitopes may also be identified by computer algorithms using conventional software programs known in the art and overlapping peptide synthesis technology, e.g., Spot Membranes (Genosys Technologies, Woodlands, Tex.) or microarray technology (JPT peptides, Berlin, Germany), or by homology to IgE-binding epitopes of other arthropods. Identification of conserved residues with epitopes from other arthropods also allows a determination of residues that cannot be substituted, as well as revealing residues that can be substituted. Accordingly, variants of the above allergens and other epitopes that maintain IgE-binding ability are also included within the disclosure.

The disclosure further provides a vector comprising an isolated nucleic acid according to the disclosure, a host cell comprising a vector, and a protein, polypeptide or peptides encoded by a nucleic acid. The vectors are useful for the expression of the nucleic acids of the invention.

The vectors of the disclosure comprise the allergen-encoding nucleic acid operably linked to suitable transcriptional and/or translational regulatory elements to effect expression in a suitable host cell. The regulatory elements may be derived from mammalian, microbial, viral or insect genes, and include, for example, promoters, enhancers, transcription and translation initiation sequences, termination sequences, origins of replication, and sequences encoding leader and transport sequences. Suitable regulatory elements are selected for optimal expression in a desired host cell. Useful expression vectors can be constructed by methods known to one of ordinary skill in the art, and are also commercially available. Exemplary recombinant viral vectors, include retrovirus, parvovirus, densovirus and baculovirus vectors.

In some embodiments, the expression vector comprises a strong constitutive or inducible promoter operatively linked to a nucleic acid of the disclosure. Suitable promoters are well known and readily available to one of ordinary skill in the art and include, for example, bacterial, yeast, viral, mammalian, and insect promoters. Exemplary expression vectors are vectors compatible with mammalian cells.

The disclosure provides host cells comprising a vector or an isolated nucleic acid as disclosed herein. Host cells comprising the vector or isolated nucleic acid are useful for replicating the vector and expressing at least the nucleic acid encoding an allergenic protein, polypeptide or peptide, or replicating and expressing the isolated nucleic acid. The host cell may be prokaryotic or eukaryotic, including bacterial, yeast, insect or mammalian cells. Exemplary host cells include insect and mammalian cells. The isolated nucleic acids or vectors, e.g., expression vectors, may be introduced into the host cells by methods known to one of ordinary skill in the art, including transformation, transfection and infection. For example, transfection may be accomplished by any known method, such as liposome-mediated transfection, calcium phosphate-mediated transfection, naked DNA transfection, microinjection or electroporation. Transformation methods suitable for prokaryotic cells are described, for example, in Cohen et al., Proc. Natl. Acad. Sci. (USA) 69:2110 (1972). Transformation of eukaryotic host cells is described, for example, in Sambrook et al., Molecular Cloning: A Laboratory Manual, 3rd edition, Cold Spring Harbor Press, Cold Spring Harbor, N.Y. (2000).

The disclosure further provides compositions comprising a protein, polypeptide or peptide comprising at least one epitope of MLC, SCP, FABP, hemocyanin or troponin C and a diluent, carrier, solubilizer, emulsifier, preservative and/or adjuvant.

The proteins, polypeptides and peptides according to the disclosure are administered as a pharmaceutical composition containing at least one such protein, polypeptide or peptide and a pharmaceutically acceptable carrier.

The protein, polypeptide or peptide may be modified and formulated for controlled delivery and for decreasing at least one undesirable clinical reaction. Methods of modifying and formulating proteins, peptides and polypeptides for immunotherapy are known to those of ordinary skill in the art. For example, U.S. Patent Publication No. US 2003/0049237 A1, incorporated herein by reference, discloses methods of encapsulating antigens to reduce association of antigen with antigen-specific IgE antibodies, thereby reducing the risk of allergic reaction and, possibly, anaphylactic shock. U.S. Patent Publication No. US 2003/0049237 A1, incorporated herein by reference, discloses methods of modifying IgE binding sites of allergens to reduce allergenicity, for example by masking the IgE binding site or altering an amino acid within the protein. International Patent Publication No. WO 00/74716 A2 discloses various carriers for peptides, as well as peptide-based vaccines in the absence of protein carriers, and compositions comprising a plurality of allergy peptides linked by an inert carrier.

The formulation of pharmaceutical compositions is generally known in the art and reference can conveniently be made to Remington's Pharmaceutical Sciences, 17th ed., Mack Publishing Co., Easton, Pa. Formulations for use in accordance with the disclosure must be stable under the conditions of manufacture and storage and must also be preserved against the contaminating action of microorganisms such as bacteria and fungi. Prevention against microorganism contamination can be achieved through the addition of one or more of various antibacterial and antifungal agents.

The pharmaceutical forms suitable for administration include sterile aqueous solutions or dispersions and sterile powders for the extemporaneous preparation of sterile injectable solutions or dispersions. In all cases, the form must be sterile and must be fluid to the extent that suitable syringability exists. Typical carriers include a solvent or dispersion medium containing, for example, water-buffered aqueous solutions (i. e., biocompatible buffers), ethanol, polyols such as glycerol, propylene glycol, polyethylene glycol, suitable mixtures thereof, surfactants, or vegetable oils.

Sterilization can be accomplished by an art-recognized technique, including but not limited to filtration or addition of antibacterial or antifungal agents, for example, paraben, chlorobutanol, phenol, sorbic acid or thimerosal. Further, isotonic agents such as sugars or sodium chloride may be incorporated in the subject compositions.

Production of sterile injectable solutions containing at least one of the subject proteins, polypeptides and/or peptides is accomplished by incorporating the compound(s) in the required amount(s) in the appropriate solvent with various ingredients enumerated above, as required, followed by sterilization, preferably filter sterilization. To obtain a sterile powder, the above sterile solutions are vacuum-dried or freeze-dried as necessary.

The subject proteins, polypeptides and/or peptides are thus compounded for convenient and effective administration in pharmaceutically effective amounts with a suitable pharmaceutically acceptable carrier and/or diluent in a therapeutically effective dose.

As used herein, the term “pharmaceutically acceptable carrier and/or diluent” includes any and all solvents, dispersion media, antibacterial and antifungal agents, microcapsules, liposomes, cationic lipid carriers, isotonic and absorption delaying agents and the like which are not incompatible with the active ingredient(s). The use of such media and agents for pharmaceutically active substances is well known in the art. Supplementary active ingredients may also be incorporated into the compositions and used in methods according to the disclosure.

It may be advantageous to formulate parenteral compositions in unit dosage form for ease of administration and uniformity of dosage. Unit dosage forms, as used herein, refers to physically discrete units suited as unitary dosages for the mammalian subjects to be treated, each unit containing a predetermined quantity of active material calculated to produce the desired therapeutic effect in association with the required pharmaceutical carrier. The specification for the novel unit dosage forms in accordance with the disclosure are dictated by, and directly depend on, the unique characteristics of the active material, and the limitations inherent in the art of compounding such an active material for the treatment of the specific allergy.

The principal active ingredient is compounded for convenient and effective administration in effective amounts with a suitable pharmaceutically acceptable carrier in unit dosage form as disclosed herein. In the case of compositions containing supplementary active ingredients, the dosages are determined by reference to the usual dose and manner of administration of the ingredient(s).

In the methods of treatment according to the disclosure that are described herein, the proteins, polypeptides or peptides may be administered in a manner compatible with the dosage formulation, in such amount as will be therapeutically effective, and in any way that is medically acceptable for the treatment of shellfish (e.g., shrimp) allergy. Possible administration routes include oral, nasal, transdermal and parenteral administration such as intravascular, intravenous, intra-arterial, subcutaneous, intramuscular, intraperitoneal, intraventricular or intraepidural. Sustained release administration is also specifically included in the disclosure.

Proteins, peptides and polypeptides may be also formulated for controlled delivery in a bacterial vector. Allergenic proteins, peptides, polypeptides or mutated hypoallergenic proteins, peptides or polypeptides are cloned in an expression vector. The vectors are transformed into, e.g., a Lactobacillus species or E. coli, and protein expression is checked by immunoblotting. Lactic acid bacteria offer the advantage of safety and a lower risk of side effects. Presentation of antigens on the surface of Lactobacilli is attractive for vaccine design, especially because the peptidoglycan layer of some strains appears to exhibit natural immunoadjuvanticity. Thus, these species are excellent candidates for the development of safe mucosal delivery vehicles of prophylactic and therapeutic molecules. Of the Lactobacillus strains previously used for vaccine delivery, L. plantarum is a good candidate. L. plantarum expressing the target protein, peptide or polypeptide is cultured in Lactobacillus medium. The cells are harvested by centrifugation, according to known protocols, and prepared for oral administration.

Shellfish allergen proteins, e.g., shrimp allergen proteins, are also used to generate antibodies. Such antibodies may be used as diagnostic and/or therapeutic agents, and include for example polyclonal, monoclonal, humanized and chimeric antibodies, single chain antibodies, antibody fragments, anti-idiotypic antibodies, and epitope-binding fragments of the foregoing antibodies. Methods of making such antibodies are known to those of ordinary skill in the art, and are disclosed, e.g., in Sambrook et al. (2000) and Harlow and Lane (1988).

Antibodies generated against the shellfish allergen proteins, peptides and polypeptides are also used to generate anti-idiotypic antibodies by methods known in the art, including, e.g., Greenspan et al., FASEB J., 7:437 (1993). Anti-idiotypic antibodies mimic the peptide and may be used for immunization. Compositions comprising an antibody or an anti-idiotypic antibody and a carrier are also provided herein.

The disclosure also provides a method of diagnosing shellfish (e.g., shrimp) allergy. The method comprises contacting an IgE-containing biological specimen or sample of a mammal with a protein, polypeptide or peptide of the invention, and detecting formation of a complex between an IgE in the specimen and the protein, polypeptide or peptide of the invention. Detection of a complex is diagnostic of shellfish (e.g., shrimp) allergy. The biological specimen or sample may be whole blood, sputum, serum, plasma, saliva, cerebrospinal fluid, urine or any other biological sample amenable to assay. Preferably the sample is a blood, serum, or plasma sample obtained from a human subject.

Immunoassay formats using proteins, peptides or polypeptides to detect specific binding partners such as antibodies in a sample are well-known in the art and are disclosed, for example, by Harlow and Lane, Antibodies: A Laboratory Manual, Cold Spring Harbor Laboratory Press, Cold Spring Harbor, N.Y. (1988). In some embodiments, the protein, peptide or polypeptide is immobilized on a solid support to bind to, e.g., an antibody specifically recognizing the immobilized molecule and to form a complex that is separated from the sample. The complex may be detected using a detection reagent that contains a reporter group, such as labeled anti-IgE. In some embodiments, the assay is an enzyme linked immunosorbent assay (ELISA). Rapid-flow-through and test-strip formats are also suitable. Competitive assays using labeled antibody may also be used.

Suitable solid supports are known in the art and include, for example, microtiter plates, nitrocellulose and other membranes, beads or discs such as glass, fiberglass, latex, polystyrene, polyvinylchloride, and magnetic particles. The proteins, polypeptides or peptides may be covalently or non-covalently attached to the support by methods known in the art.

The disclosure also provides methods for treating a shellfish (e.g., shrimp) allergy comprising administering a composition comprising a therapeutically effective amount of a protein, polypeptide, peptide, analog, derivative or fragment thereof in accordance with the disclosure to a mammal in need of such treatment. In some embodiments, the mammal is a human. Analogous immunotherapeutic methods are known to those of ordinary skill in the art. A therapeutically effective amount of the protein, polypeptide, peptide, analog, derivative or fragment thereof is defined herein as an amount effective to achieve hyposensitization. The precise therapeutically effective amount of the protein, polypeptide, peptide, analog, derivative or fragment thereof to be used in a method of treatment according to the disclosure, e.g., a method of treating a human, can be determined by the ordinary skilled artisan with consideration of individual differences in age, weight, extent of disease and condition of the patient.

The disclosure also provides kits useful for the detection of shellfish (e.g., shrimp) allergy. The kits comprise one or more proteins, polypeptides or peptides according to the disclosure, and an agent for detecting a complex of an antibody and the protein, peptide or polypeptide. In some embodiments, the protein, peptide or polypeptide is immobilized.

In accordance with the disclosure, several shellfish, e.g., shrimp, allergens have been identified that can be utilized in the diagnosis and treatment of patients with shellfish (e.g., shrimp) allergy. The proteins identified as shellfish (e.g., shrimp) allergens are myosin light chain, (MLC) sarcoplasmic calcium-binding protein (SCP), hemocyanin, shrimp fatty acid binding protein (FABP) and troponin C derived from shellfish, e.g., shrimp.

The nucleotide sequences encoding shrimp myosin light chain and shrimp sarcoplasmic calcium binding protein have been submitted to the GenBank database under Accession Numbers EU449515 and FJ184279, respectively. The World Health Organization/International Union of Immunological Societies Allergen Nomenclature Subcommittee has designated shrimp MLC as Lit v 3 and shrimp SCP as Lit v 4.

The protein, polypeptides and peptides of shrimp myosin light chain (Lit v 3), shrimp sarcoplasmic calcium-binding protein (Lit v 4), shrimp hemocyanin, shrimp FABP and shrimp troponin C are recognized by serum IgE from patients with shrimp allergy.

The disclosure provides isolated nucleic acids that encode shrimp myosin light chain (Lit v 3), shrimp sarcoplasmic calcium-binding protein (Lit v 4), shrimp hemocyanin, and shrimp FABP, that are recognized by antibodies specific for shrimp allergens.

The following examples illustrate embodiments of the invention. Example 1 describes methods used in the work described throughout the disclosure; Example 2 discloses the immunoreactivity of serum Immunoglobulin E to shrimp extracts; Example 3 discloses immunoblot inhibition by shellfish troposmyosin; Example 4 describes the identification of shellfish (shrimp) allergenic protein; Example 5 describes the cloning and sequencing of shrimp SCP; Example 6 details the binding capacity of shrimp rSCP to IgE and its cross-reactivities; Example 7 discloses ELISA inhibition assays of the shellfish allergenic protein; and Example 8 reveals the capacity of rSCP to induce mediator release.

### EXAMPLE 1

### Methods

Patient Selection

For the studies relating to shellfish myosin light chain polypeptide, sera were obtained from 38 patients with shrimp allergy (18 [47.3%] adult and 20 [52.6%] pediatric patients) with histories of immediate allergic reactions after the ingestion of shrimp and increased serum IgE levels to shrimp. Specific IgE levels to shrimp were determined by using UniCAP (Phadia, Uppsala, Sweden) and considered positive if greater than 0.35 kU/L. Levels of specific serum IgE to other arthropods, the house dust mite Dermatophugoides pteronyssinus, and the cockroach Blattella germanica, were also included. Patient sera were collected from Hospital del Nino Jesus, Madrid, Spain (subjects 1-10); Hospital Universitario Dr. Negrin, Las Palmas de Gran Canaria, Spain (subjects 17-37); and Mount Sinai Medical Center, New York (subjects 11-16 and 38; Table I). This study was approved by the institutional review board of Mount Sinai Medical Center.

TABLE I
Clinical characteristics, type of reaction with shrimp, and specific serum
IgE values of the 38 study subjects

| | | | | Shrimp | B germanica | D pteronyssinus |
|---|---|---|---|---|---|---|
| Patient no. | Age (y) | Diagnosis | Shrimp reaction | IgE (kU/L) | IgE (kU/L) | (kU/L) |
| 1 | 10 | A, AR, FA | U | 49.5 | 12 | 9.5 |
| 2 | 17 | A, AR, FA, AD | Inh-A, AE | 52.1 | 12 | 9.7 |
| 3 | 10 | A, AR, FA, AD | U | 11.3 | 6.3 | 3.8 |
| 4 | 12 | A, AR | U, AE | 64.3 | 28 | 13.5 |
| 5 | 13 | A, AR, RA | Inh-A, AE | 82.4 | 43 | 22.4 |
| 6 | 12 | A, AR, FA, AD | Inh-A, U | 74.3 | 32 | 12.1 |
| 7 | 8 | A, AR, FA, AD | U | 0.38 | 0.4 | 0.6 |
| 8 | 12 | AR | AE, RC | 47 | 32 | 18 |
| 9 | 11 | FA | U | 3.6 | 0.9 | <0.38 |
| 10 | 7 | FA | U | 14 | 3.6 | 5.3 |
| 11 | 29 | AR, FA | RC, AB | 10 | 1.8 | 1.0 |
| 12 | 6 | AD, FA, A, AR | AE, U, OI, CU | 1.9 | 3.4 | 4.7 |
| 13 | 3 | AD, FA | U | 100 | 74 | >100 |
| 14 | 8 | AD, A, AR | GI, RC, U | 100 | 30 | 23 |
| 15 | 6 | AD, FA, A | OAS | 8 | 5.3 | >100 |
| 16 | 6 | AR, A | AB, U, A | 30 | 42 | 7.1 |
| 17 | 38 | A/AR | AB, U, CU | >100 | 12.5 | >100 |
| 18 | 34 | AR | AE, U | >100 | <0.35 | >100 |
| 19 | 16 | A/AR/FA | ANA | >100 | 8.9 | >100 |
| 20 | 27 | | OAS | 93 | 4.2 | >100 |
| 21 | 33 | AR/A | AE, A | 65 | 22.6 | 40.3 |
| 22 | 32 | AR | AE, RC | 48 | 12.3 | 59 |
| 23 | 15 | AR | AE | 55 | 23 | >100 |
| 24 | 11 | A/AR | U, GI | 5 | 1 | >100 |
| 25 | 40 | A/AR | AB, A | 14 | 3.2 | 9 |
| 26 | 13 | | U | 0.8 | <0.35 | >100 |
| 27 | 24 | A/AR | OAS | 0.36 | <0.35 | 77 |
| 28 | 34 | | ANA, A, AH | 1 | 2.4 | 1.1 |
| 29 | 19 | | AE, RC | 10 | 3 | 8.4 |
| 30 | 19 | A/AR | AB | 1 | <0.35 | 56 |
| 31 | 26 | A/AR | AE | 3.7 | 3.8 | >100 |
| 32 | 32 | AR | AB, U | 8 | 2 | 34.6 |
| 33 | 46 | FA | AE, U, A | 0.6 | 1 | 21 |
| 34 | 26 | AR | OAS | 2 | 1.5 | >100 |
| 35 | 19 | FA | U | 14 | 8 | >100 |
| 36 | 17 | AR | AE, U | 11 | 0.6 | 55 |
| 37 | 27 | AR | AE | 6 | 2.8 | 77 |
| 38 | 30 | AR | AE | 15 | 35 | 24.5 |

[symbol]

[symbol]

indicates data missing or illegible when filed

For experiments relating to sarcoplasmic calcium-binding protein, sera were obtained from 52 subjects with shrimp allergy, 23 children (44.2%) age 3 to 18 years (mean, 10.2 years) and 29 adults (55.7%) age 19 to 70 years (mean, 30.9 years), with immediate allergic reactions after the ingestion of shrimp, and elevated serum IgE to shrimp. Subjects 1-38 were characterized and used in the studies relating to myosin light chain polypeptide, as described above, and 14 additional patients were recruited (9 children and 5 adults). Shrimp-specific IgE levels were determined by Uni-CAP (Phadia, Uppsala, Sweden) and again considered positive if greater than 0.35 kUA/L. As with the MLC studies, patients' sera were collected from the Hospital del Nino Jesus, Madrid, Spain; Hospital Universitario Dr Negrin, Las Palmas de Gran Canaria, Spain; and Mount Sinai Medical Center, New York. Patient characteristics did not differ significantly from those described in Ayuso et al., J. Allergy Clin. Immunol. 122:795-802 (2008), incorporated herein by reference.

Shellfish Extract Preparation

For myosin light chain polypeptide studies, extracts were prepared from raw tail muscle of the white leg Pacific shrimp (L. vannamei). Raw peeled and deveined shrimp tail muscle was manually homogenized in a mortar until a smooth paste was achieved. Protein was extracted by means of agitation in PBS with a protease inhibitor cocktail without ethylenediamine tetra-acetic acid (Roche, Indianapolis, Ind.). NaN₂ in distilled water (20% wt/vol) was added (1:400) as preservative and incubated overnight at 4° C. The mixture was centrifuged at 3000 rpm for 10 minutes at 4° C. and then at 15,000 rpm for 5 minutes at 4° C.

Extract of boiled shrimp was prepared by boiling peeled, deveined shrimp tail muscle for 5 minutes in distilled water and homogenized according to the same protocol as above. Protein concentration was determined by means of spectrophotometry with the Coomassie Plus Protein Assay (Pierce, Rockford, Ill.). Extracts were stored at −20° C.

Extract of boiled shrimp used for protein sequencing was prepared from peeled fresh market shrimp. PBS was added for protein extraction and incubated overnight at 4° C. Homogenized paste was centrifuged at 3000 rpm for 20 minutes at 4° C. The supernatant was collected and recentrifuged for 20 minutes and stored at −20° C. until use.

For inhibition studies, chickpea (Cicer aretimon) extract was used as the control. Briefly, chickpeas purchased locally at an Indian supermarket were boiled in water for 30 minutes. Cooked chickpeas were homogenized, and protein was extracted as described for shrimp.

For experiments relating to SCP, extracts were prepared from raw and boiled tail muscle of the Pacific white shrimp (L. vannamei) as described above and in Ayuso et al. 2008, incorporated herein by reference. Raw crab abdominal muscle, lobster tail, squid, mussel, and scallop extracts were boiled for 5 minutes in distilled water and manually homogenized in a mortar. Protein was extracted by agitation in PBS with protease inhibitor cocktail without EDTA (Roche, Indianapolis, Ind.). Sodium azide in distilled water (20% wt/vol) was added (1:400) as preservative and incubated overnight at 48° C. The mixture was centrifuged at 48° C. at 3000 rpm for 10 minutes and then at 15,000 rpm for 5 minutes. The protein concentration was determined with a Coomassie Plus Protein Assay (Pierce, Rockford, Ill.). Extracts were stored at −20° C. Chick pea extract was prepared as described (Ayuso et al., 2008, incorporated herein by reference).

SDS-PAGE and 2-Dimensional Analysis

For SDS-PAGE analyses, samples were heated at 70° C. for 10 minutes with Nu-page sample buffer in the presence of 0.05 mol/L dithiothreitol. Proteins were then separated by means of SDS-PAGE (Nupage 4-12% Zoom Gels; Invitrogen, Carlsbad, Calif.), according to the manufacturer's instructions. Protein was loaded onto the gel at a concentration of 12.5 μg/cm of gel.

For 2-dimensional (2-D) electrophoresis, protein extract was suspended in rehydration buffer. See Beyer et al., J. Allergy Clin. Immunol. 110:154-159 (2002) (“Beyer et al. (2002a)”), incorporated herein by reference. Then 25 μg of shrimp extract was applied to immobilized pH 4 to 7 (7-cm) gradient strips (Bio-Rad, Hercules, Calif.) for rehydration and focusing, as described in Beyer et al. (2002a). Strips were equilibrated, as described by Beyer et al. (2002a), and run with SDS-PAGE.

Gels were either stained with Simply Blue SafeStain (Invitrogen), according to the manufacturer's protocol, or proteins were transferred onto Immobilon-P membranes (Millipore, Bedford, Mass.), as described in Beyer et al. (2002a). Membranes were stained with amido black 0.1% (10% methanol and 2% acetic acid) staining solution or tested for IgE binding with patients' sera.

Protein identification was done from 2-D gels stained with Simply Blue SafeStain. Proteins of interest were analyzed at the Wistar Institute Protein Microchemistry/Mass spectrometry Facility, as described in Beyer et al. (2002a). Proteins of interest were excised, and “in-gel” digestion was performed. The tryptic digests were separated by means of HPLC, followed by matrix-assisted laser desorption/ionization (MALDI)/mass spectrometric (MS) analysis of selected peaks and subsequent Edman sequencing of selected peaks.

For SCP studies, proteins were separated by SDS-PAGE (Nupage 4% to 12% Zoom Gels; Invitrogen, Carlsbad, Calif.) following the manufacturer's instructions. Protein was loaded at a concentration of 12.5 μg protein/cm gel. Two-dimensional electrophoresis was performed as described in Beyer et al., (2002a), incorporated herein by reference. Gels were stained with Simply Blue SafeStain (Invitrogen), or proteins were transferred onto Immobilon-P membranes (Millipore, Bedford, Mass.) as described above. Membranes were stained with 0.1% Amido Black (10% methanol, 2% acetic acid) staining solution or tested for IgE binding with patients' sera.

Protein identification was performed from 1-dimensional and 2-dimensional gels stained with Simply Blue SafeStain (Beyer et al., 2002a). A 20-kd protein was excised, and in-gel digestion was performed. Sequence analysis of tryptic digests of the spot of interest (from a 2-dimensional gel) was performed at the Wistar Institute Proteomics Facility using microcapillary reverse-phase HPLC nano-spray tandem mass spectrometry on a ThermoFinnigan LTQ quadrupole ion trap mass spectrometer. The mass spectrometer measures peptide masses and then fragments individual peptides to produce MS/MS spectra of fragments that reflect the peptide sequence. The MS/MS spectra are run against a nonredundant sequence database (NCBI) using the program SEQUEST. If greater than or equal to three peptide sequences in a database entry were matched by MS/MS spectra as disclosed herein, the protein identification had a high confidence level.

Immunoblot Analysis with Sera from Subjects with Shellfish Allergy

Immunoblot detection for IgE binding in the MLC studies was done with extracts of raw and boiled L. vannamei. Membranes were incubated with sera from 38 patients with shrimp allergy (1:5 to 1:20 in PBS-Tween [1% BSA and 10% normal goat serum]) for 1 hour. After rinsing with PBS, the membranes were incubated with iodine 125-labeled goat anti-human IgE (DiaMed, Windham, Me.) diluted according to the manufacturer's instructions, washed, and exposed to Kodak Imaging Film (Rochester, N.Y.) for 1 to 12 days. As a negative control, serum was used from a nonatopic subject. IgE-binding proteins in 2-D immunoblots were visualized with phosphatase-labeled goat anti-human IgE, as described in Beyer et al. (2002a).

For immunoblot inhibition experiments, a serum pool was prepared from seven subjects with IgE antibodies that recognized a 20-kd shrimp protein by means of immunoblotting. Then 150 μL. of the diluted serum pool (1:20 in PBS-Tween) were preincubated at room temperature for two hours with recombinant tropomyosin at a final concentration of 25 μg/mL. The mix was incubated with the shrimp membranes, as described above. As a control, the pool was preinhibited with chickpea extract at a final concentration of 1.5 mg/mL.

For SCP studies, immunoblots for detection of IgE binding were also performed with extracts of raw and boiled L. vannamei. Membranes were incubated with sera from patients with shrimp allergy (1:5 to 1:20 in PBS-Tween [1% BSA, 10% normal goat serum]) for 90 minutes. After rinsing with PBS, the membranes were incubated with iodine 125-labeled goat antihuman IgE (DiaMed,Windham, Me.) for one hour, diluted per manufacturer's instructions, washed, and exposed to Kodak Imaging Film for 1 to 12 days. As negative control, sera from 2 non-atopic subjects were used.

Molecular Cloning of Shellfish Protein-Encoding Nucleic Acids

A cDNA library was generated from raw pacific white shrimp, L. vannamei. Total RNA was extracted and mRNA purified on Oligotex mRNA Spin-column (Quiagen, Valencia, Calif.). Synthesis and cloning of cDNA was done with a cDNA synthesis kit from Stratagene (La Jolla, Calif.). The double-stranded cDNA was ligated into the lambda vector Uni-ZAP, packaged in vitro (Gigapack Gold; Stratagene), and transfected into Escherichia coli XE1-Blue cells. See Beyer et al., J. Allergy Clin. Immunol. 110:517-523 (2002) (“Beyer et al. (2002b)”), incorporated herein by reference.

The library was screened with primers designed on the basis of internal sequences obtained from 2-D tryptic digests. For screening of the cDNA library, two degenerate oligonucleotides were used 5′TTYGAYATGTTYACICARA3′ (SEQ ID NO: 28) and 5′AACGTGTTCGACATGTTCACCCAGAAG3′ (SEQ ID NO: 29), resulting in isolation of a novel protein cDNA. Open reading frame translation of this cDNA, sequenced at the Mount Sinai Core Facility, provided the complete amino acid sequence of a new allergenic shrimp protein.

The cDNA library was also used to obtain cDNA encoding sarcoplasmic calcium-binding protein (SCP) by means of PCR in two steps. First, two 5′-specific primers were designed based on sequences from Penaeus monodon SCP (AI253941; 5′-TATATGTACGACATTGACAAC-3′ (SEQ ID NO: 19) and 5′-GATAAGAACGACTTCGAGTGC-3′ (SEQ ID NO: 20)) encoding the peptides YMYDIDN (SEQ ID NO: 21) and DKNDFEC (SEQ ID NO: 22), respectively, identical to the homologous sequences in SCP α-B and α-A chains, and in the SCP β chain from Penaeus species (P02636, P02635). M13 Forward (−20) was used as the 3′-primer specific end for the cDNA library vector. PCR product was cloned into pCR2.1-TOPO vector (TOPOTA cloning kit; Invitrogen) and sequenced at the Mount Sinai Core Facility. Second, the missing 5′ end of the cDNA was amplified by PCR using the specific reverse primers (5′-TTGAACTGGTTGGCAATGAA-3′ (SEQ ID NO: 23) and 5′-GTAAGCGTCATCAATCTCATTC-3′ (SEQ ID NO: 24)) based on the internal sequence from the cloned L. vannamei SCP cDNA obtained in the previous step, and the T3 forward primer from the cloning vector. The PCR product was ligated into TOPO vector and sequenced. The sequence analysis was performed with Vector NTI Advance 10 (Invitrogen) software.

Production of Recombinant Protein

Recombinant proteins were obtained under native conditions, as described in Beyer et al., (2002b). Briefly, the protein-coding regions of tropomyosin and myosin-like protein were amplified by means of PCR, with specific primers 5′ TTTTTGGATCCGATGTCCCGCAAGTCAGGCT (SEQ ID NO: 54) and 3′ TTTTTCTCGAGGGCTTCCTCTGCGGCAGCGT (SEQ ID NO: 55), for ligation into the expression vector pET24b(+) (Novagen, Madison, Wis.). The vector-plasmid construct was then transformed into E. coli according to the manufacturer's instructions (Stratagene). The plasmids were then purified, and the nucleotide sequence was confirmed. Each individual plasmid was introduced into the BL21 expression E. coli strain. The recombinant proteins were expressed after induction of bacterial cultures at 30° C. for 16 hours and detected by means of Western blot analyses with anti-His-Tag antibodies. Finally, recombinant proteins were purified with a His-Bind Ni²⁺-chelating NTA-matrix resin (Novagen).

For SCP experiments, the protein-coding region of SCP was amplified by PCR with specific primers, forward with Hind III restriction site (5′-AAAAAA GCT TAT GGC TTA CAG TTG GGA CA-3′ (SEQ ID NO: 25)) and reverse with Xho I site (5′-TAT TTC TCG AGC TGC ACC ACC TTC AGG GG-3′ (SEQ ID NO: 26)), and ligated into the expression vector pET24b(1) (Novagen, Madison, Wis.). The vector-plasmid construct was transformed into Escherichia coli XL1 Blue strain according to the manufacturer's instructions (Stratagene, La Jolla, Calif.). The plasmid was purified and introduced into the BL21 expression strain. Recombinant (r) SCP was expressed after induction of bacterial cultures with isopropyl β-D-thiogalactoside at 37° C. for 16 hours, and detected by Western blot analyses with anti-His-Tag antibodies. Recombinant protein was purified with a His-Bind Ni²⁺-chelating NTA-matrix resin (Novagen) under native conditions. Recombinant shrimp tropomyosin (rLit v 1) and recombinant hazelnut 11S storage protein (rCor a 9) were obtained from the Mount Sinai Food Allergen Repository.

Probing Recombinant Proteins with Subjects' Sera

Recombinant MLC was tested for IgE reactivity with individual sera from 19 patients with shrimp allergy that recognized a 20-kd protein by means of immunoblotting. A nonatopic adult was used as a negative control. Recombinant protein was loaded onto the gel at a concentration of 2.8 μg of protein/cm of gel. The remainder of the protocol is as described above in the SDS PAGE and immunoblotting sections. Membranes were incubated with iodine 125-labeled goat anti-human IgE and exposed to Kodak Imaging Film for 12 days.

Recombinant SCP was tested for IgE reactivity by immunoblotting with sera from 31 subjects with shrimp allergy that recognized a 20-kd protein. Three nonatopic adults were used as negative controls. Recombinant protein was loaded at a concentration of 2.5 μg protein/cm gel.

For immunoblot inhibition experiments, a serum pool was prepared from subjects 2 and 47, which recognized rSCP by immunoblot analysis with high intensity. Diluted serum pool (1/20 in PBS-Tween) was preincubated at room temperature for two hours with inhibitor at a concentration of 0.5 mg/mL. Extracts of dust mite Dermatofagoides farinae (Greer Lab Inc, Lenoir, N.C.), German cockroach Blatella germanica (Greer Lab Inc), BS (boiled shrimp), lobster tail muscle, crab, squid, scallop, and mussel were used as inhibitors, and chickpea extract as a negative control. The mix was then incubated with the membrane-containing rSCP as described.

ELISA Inhibition

For ELISA inhibition studies, 96-well plates were coated overnight at 48° C. with 100 μL per well of BS extract (25 μg/mL) in carbonate-bicarbonate buffer 0.05 mol/L, pH 9.6 (Sigma-Aldrich, St Louis, Mo.). Sera from subjects 2 and 43, which recognized rSCP by immunoblotting, were diluted 1:50 and 1:20, respectively, in 2% BSA, 0.05% Tween phosphate buffer saline (PBST), were separately preincubated without inhibitor and with increasing concentrations (0.25 pg/mL to 25 μg/mL) rSCP, rLit v 1, BS extract, or rCor a 9 (as negative control) for one hour at 37° C. Inhibition samples were added to the plate and incubated for 2 hours at room temperature. Allergen-specific IgE was detected with horseradish peroxidase-labeled goat antihuman IgE (KPL, Gaithersburg, Md.) 1:2500, developed with tetramethylbenzidine peroxidase substrate (KPL), and read on a microplate reader at 650 nm. The percent inhibition values were calculated as follows: ((OD uninhibited−OD inhibited)/(OD uninhibited−OD buffer))×100.

Mediator Release Assay

The mediator release assay was performed as described (Hoffmann et al., Allergy 54:446-54 (1999); Kaul et al., ALTEX 18:55-8 (2001), each incorporated herein by reference) with rat basophilic leukemia (RBL)-2H3 cell line transfected with human Fcε receptor type 1. RBL cells were maintained in 75% Eagle's Minimal Essential Medium (MEM) and 15% RPMI (Cellgro; Mediatech Inc, Herndon, Va.) with 10% FCS (HyClone, Logan, Utah) and G418 sulfate (Acros Organics, Fair Lawn, N.J.). RBLs were sensitized with pooled sera (1:20) of three patients and a nonatopic human serum in 96-well tissue-culture plates (BD Falcon, Bedford, Mass.) at 37° C. in 5% CO₂ for 18 to 20 hours. Cells were stimulated for one hour at 37° C. in 5% CO₂ with 100 μL per well of 1 μg/mL rSCP, rLit v 1, and BS extracts, followed by 10-fold serial dilutions in a release buffer containing D₂O (Acros Organics). Rabbit IgG antihuman polyclonal IgE (Bethyl Laboratories Inc., Montgomery, Tex.) was used as a positive control for IgE-mediated degranulation. β-hexosaminidase was measured as a marker for mediator release. To determine β-hexosaminidase release, 30 μL supernatant was gently mixed with 50 μL P-nitrophenyl-N-acetyl-β-D-glucosaminide (pH 4.5; Sigma-Aldrich, St Louis, Mo.) for one hour. The reaction was terminated by adding 100 μL 0.2 mol/L glycine solution (pH 10.7), and absorbance at 405 nm was measured. Total mediator release was obtained by lysing the cells with 1% Triton X-100 (Sigma). Results were expressed as percentage of release from cells sensitized with serum minus spontaneous release, divided by total release.

### EXAMPLE 2

IgE Reactivity of Subjects' sera to L. vannamei Extracts

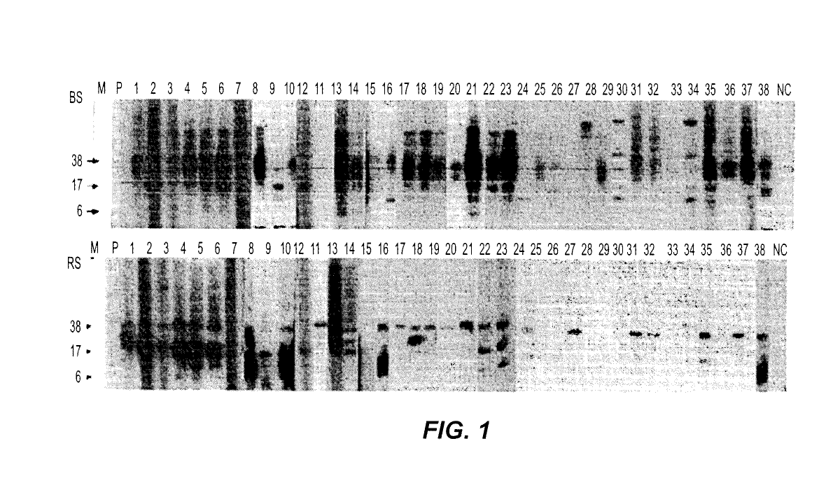

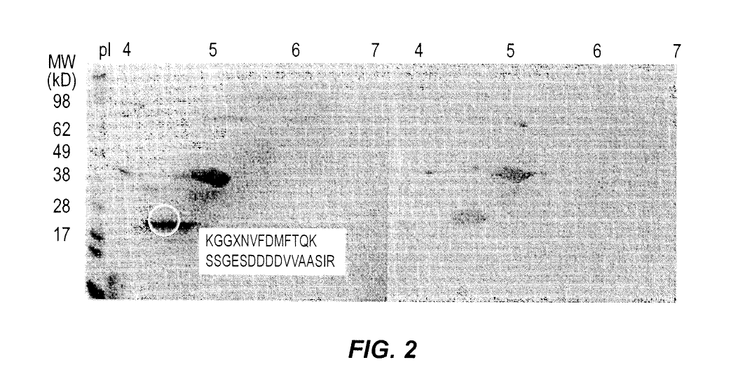

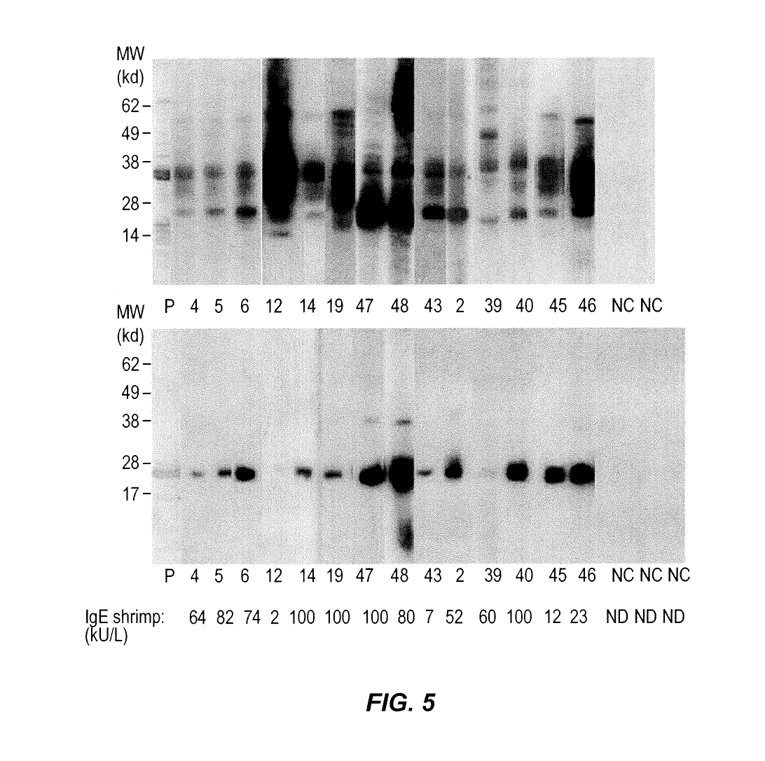

Subjects with shrimp allergy show IgE reactivity by means of immunoblotting to multiple shrimp proteins ranging from 6 to 90 kd (FIGS. 1 and 5A). Although boiled shrimp extracts have multiple allergenic proteins, in raw extracts IgE reactivity is mostly directed at tropomyosin and a limited number of proteins of less than 40 kd. Among all proteins, a 20-kd protein was recognized in boiled extracts with particular intensity by 21 (56.6%) of 37 sera used in MLC studies. Most subjects recognized a protein of similar molecular weight in raw extracts but generally with lower intensity, except for a group of pediatric patients (subjects 2-8) who showed increased IgE binding to the raw 20-kd protein. In several cases this protein was recognized with higher intensity than the 34-kd band corresponding to tropomyosin. Interestingly, for subjects 9 and 12, this was the only protein recognized in both raw and boiled extracts (FIG. 1). Immunolabeling of 2-D membranes of boiled shrimp with serum from subject 38 demonstrated intense IgE binding to a 20-kd shrimp protein, with an isoelectric point of 4.2 (FIG. 2). The control sera did not show any labeling.

A 20-kd protein was recognized in boiled extracts with particular intensity by 31 of 52 (59.6%) sera (see FIG. 5) used in SCP experiments. In several cases, this protein was recognized with higher intensity than the 34-kd band corresponding to tropomyosin (FIG. 5A). Two control sera did not show any labeling.

### EXAMPLE 3

Immunoblot Inhibition with Recombinant Tropomyosin

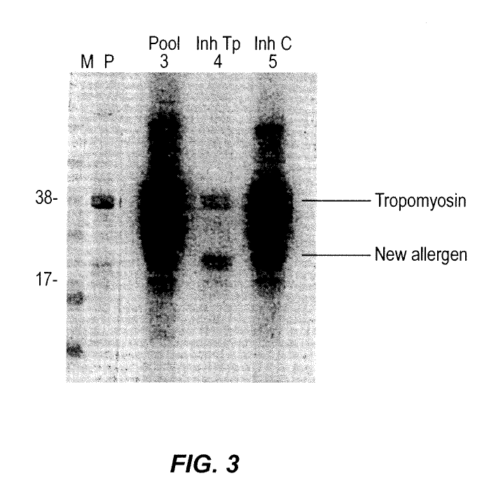

A pool of patients' serum (MLC studies) was preabsorbed with recombinant tropomyosin to determine whether this was a new allergenic shrimp protein. Preincubation of the serum pool with tropomyosin almost completely abolished IgE binding to most shrimp proteins. IgE reactivity to the 20-kd protein, however, was not inhibited, indicating that this is a novel shrimp allergen (FIG. 3). Inhibition of IgE binding to the 34-kd protein tropomyosin was incomplete, possibly because of an insufficient amount of inhibitor or because of the presence of IgE-binding dimers of the 20-kd protein.

### EXAMPLE 4

Protein Identification by LC-MS/NIS Analysis of Tryptic Digests from a 2-Dimensional Gel

The 20 kd protein identified as a shrimp allergen in the immunoblot inhibition study of Example 3 was subjected to sequence analysis. Peptide fragments resulting from a tryptic digestion of the 20 kD protein from a 2-D gel were separated by means of microbore HPLC and MALDI/MS analysis of the peptides at the Wistar Facility, followed by Edman sequencing of 2 selected peaks. The analysis identified the protein as a myosin light chain (FIG. 2). The sequences of two peptides were obtained: KGGXNVFDMFTQK (SEQ ID NO: 1) and SSGESDDDDVVAASIR (SEQ ID NO: 2). The identified peptides showed high sequence identity with the homologous sequence of another allergenic MLC molecule, the German cockroach protein Bla g 8.

Some subjects who recognized a 20-kd protein by IgE immunoblotting showed little binding to MLC (i.e., rLit v3), indicating the presence of another allergenic protein of similar molecular weight. Tryptic digests of this additional 20 kD protein, identified as a spot of interest on a 2-D gel, led to the generation of various peptide fragments. These peptide fragments were analyzed by LC-MS/MS and the amino acid sequences of these peptide fragments (see Table II) led to the identification of the protein in question as an SCP. Columns 1-3 of Table II provide data relating to the SCP a chain (Accession No. P02636), for which sequence coverage was 59%. Columns 4-6 of Table II provide data relating to the SCP β chain (Accession No. P02635), for which sequence coverage was 31%. A number of the 17 peptides matched SCP α-B and α-A chains (P02636), with sequence coverage of 59% and a spectral count of 187. Seven peptides matched SCP β chain (P02635), with sequence coverage of 31% and a spectral count of 90. Results for LC-MS/MS analyses of 1-dimensional gels were similar to those from 2-dimensional gels.

TABLE II
Protein identification by LC-MS/MS analysis of tryptic
digests from a 2-dimensional gel

| Peptide | | | SEQ | Peptide | | | SEQ |
|---|---|---|---|---|---|---|---|
| sequence | Peptide | Sequest | ID | sequence | Peptide | Sequest | ID |
| (SCP α) | position | score | NO | (SCP β) | position | score | NO |
| YMYDIDDDGFLDK | 14-26 | 0.92 | 30 | YMYDIDNDGFL | 14-26 | 0.90 | 47 |
| | | | | DK | | | |
| YMYDIDDDGFLDK | 14-35 | 0.88 | 31 | YMYDIDNDGFL | 14-25 | 0.92 | 48 |
| NDFLCLAVR | | | | DKNDFECLAVR | | | |
| NDFECLAVR | 27-35 | 0.91 | 32 | NDFECLAVR | 27-35 | 0.91 | 49 |
| GEFSAADYANNQ | 43-55 | 0.90 | 33 | DGEVTVDEFK | 73-82 | 0.88 | 50 |
| K | | | | | | | |
| NLWNEIAELADFN | 59-82 | 0.86 | 34 | DGEVTVDEFKQ | 73-87 | 0.90 | 51 |
| KDGEVTVDEFK | | | | AVQK | | | |
| NLWNEIAELADFN | 59-72 | 0.96 | 35 | EIDDAYDK | 140-147 | 0.30 | 52 |
| K | | | | | | | |
| DGEVTVDEFK | 73-82 | 0.88 | 36 | YQELYAQFISNE | 164-178 | 0.99 | 53 |
| | | | | DEK | | | |
| VFIANQFKAIDVN | 103-119 | .87 | 37 | | | | |
| GDGK | | | | | | | |
| VFIANQFK | 103-110 | 0.75 | 38 | | | | |
| AIDVNGDGK | 110-119 | 0.53 | 39 | | | | |
| VGLDEYR | 120-126 | 0.72 | 40 | | | | |
| SAFAEVKEIDDAY | 133-147 | 0.95 | 41 | | | | |
| NK | | | | | | | |
| EIDDAYDK | 140-147 | 0.30 | 42 | | | | |
| EIDDAYDKLTTED | 140-155 | 0.71 | 43 | | | | |
| DRK | | | | | | | |
| LTTEDDRK | 148-155 | 0.54 | 44 | | | | |
| KAGGLTLER | 155-163 | 0.79 | 45 | | | | |
| AGGLTLER | 156-163 | 0.80 | 46 | | | | |

*Sequest score range, 0 to 1. Example 5

### Cloning and Sequencing of Shellfish Protein Allergens

A cDNA library was prepared from fresh white pacific shrimp, L. vannamei. The shrimp cDNA library was screened with primers designed on the basis of the internal sequence obtained from one of the 2-D tryptic digests (KGGXNVFDMFTQK (SEQ ID NO: 27)). The degenerated oligonucleotides 5′TTYGAYATGTTYACICARA3′ (SEQ ID NO: 28) and 5′AACGTGTTCGACATGTTCACCCAGAAG3′ (SEQ ID NO: 29) were used as primers, resulting in isolation of a shrimp cDNA. Open reading frame translation of this cDNA provided the complete amino acid sequence of a new allergenic shrimp protein. The new allergenic shrimp protein, referred to herein as myosin light chain (MLC), was named Lit v 3.0101 by the international Allergen Nomenclature Committee.

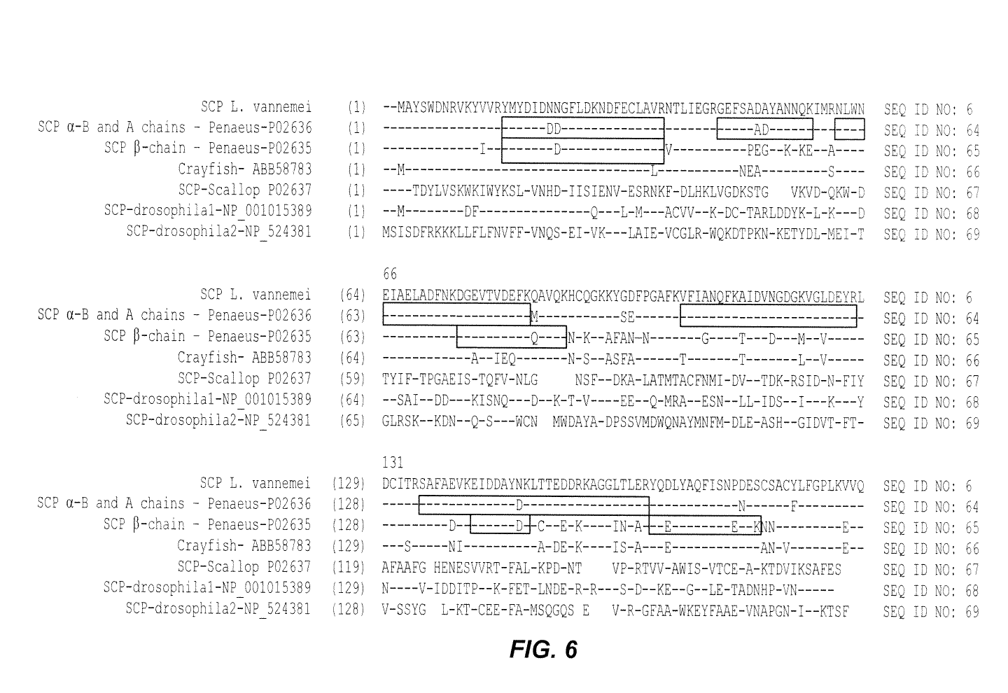

The SCP gene was amplified from the shrimp cDNA library by PCR with two forward specific primers and a reverse primer, M13. A few clones for each PCR product were sequenced, all encoding the same SCP. Because the 5′ end of the open reading frame encoding the N-terminal part of the protein was absent, gene-specific reverse primers and T3 primer were used to obtain the missing 5′ end of the cDNA by PCR. Sequence analysis of these PCR products provided the complete cDNA encoding SCP from L. vannamei (GenBank Accession No. FJ184279). This SCP protein was designated Lit v 4.0101 allergen by the International Allergen Committee. The deduced amino acid sequence of Lit v 4.0101 and other SCP molecules are aligned in FIG. 6, with the percent amino acid identity between them apparent from inspection of FIG. 6 in view of the brief description of that drawing. The highest amino acid identity is seen with crustacean SCPs (80% to 93%), whereas identity with other arthropod and mollusk SCPs was low.

### EXAMPLE 6

IgE Binding Capacity of Recombinant Shellfish Allergen Proteins and Cross-Reactivity with Other Arthropods and Mollusks

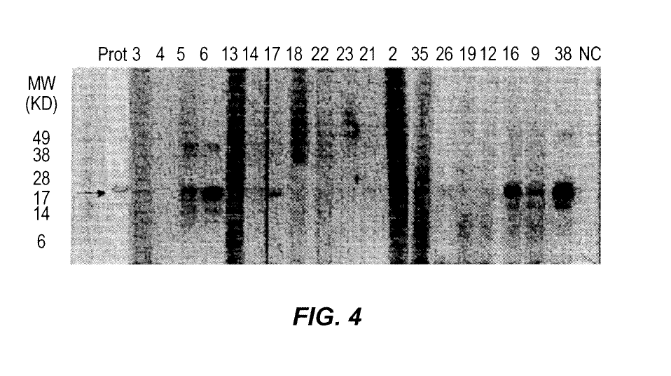

Recombinant shrimp MLC was tested for IgE binding with individual sera from the 19 patients (MLC studies) who recognized a 20-kD protein by means of immunoblotting. Most (17/19 of the tested subjects recognized the recombinant shrimp MLC, although with weaker intensity than the native protein (FIG. 4).

Recombinant shrimp SCP was also tested for IgE binding with individual sera from 31 of 52 subjects (22 children and 9 adults) that recognized a 20-kd protein by immunoblot. Twenty of 31 (64.5%) subjects tested, representing 38.4% of the total (20/52), recognized the rSCP by immunoblot. Those sera not recognizing rSCP by immunoblot did not recognize native 20 kD SCP and, thus, lacked anti-SCP IgE. Interestingly, 17 of 20 (85%) of these subjects were children. A sample of subjects with the highest IgE reactivities is shown in FIGS. 5A and 5B (all the subjects are children except subject 39, who is an adult).

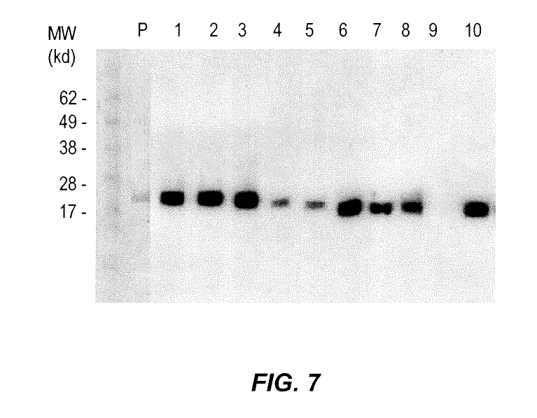

For immunoblot inhibition experiments, a pool of subjects' sera was preabsorbed with extracts from cockroach, dust mite, boiled crab, lobster, squid, scallop, and mussel as inhibitors, and chick pea as control. Inhibition with lobster and crab significantly decreased IgE binding to rSCP, suggesting cross-reacting epitopes among crustacean SCPs (FIG. 7). In contrast, preincubation with dust mite (D. farinae), cockroach (B. germanica), or mollusks did not show significant inhibition. BS completely inhibited IgE reactivity to the rSCP, whereas chickpea extract used as negative control showed no inhibition.

### EXAMPLE 7

### ELISA Inhibition

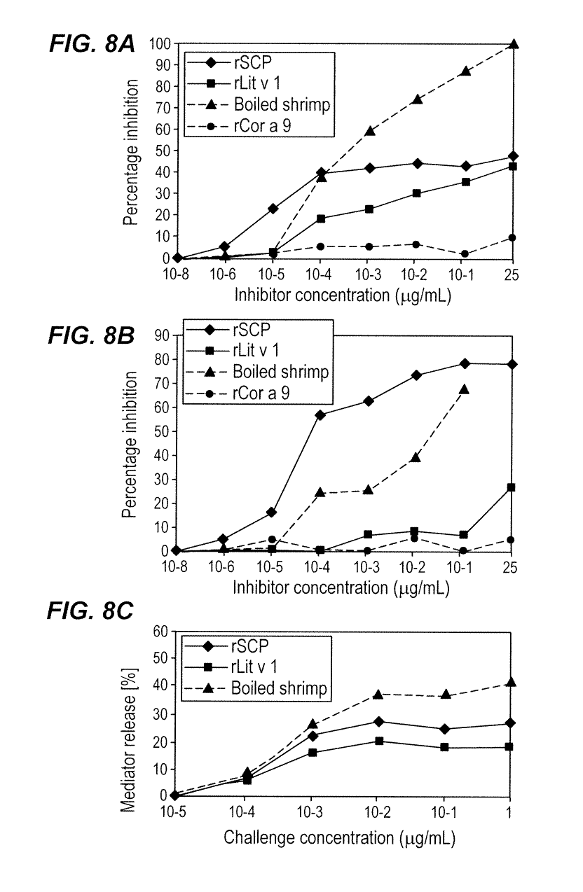

For ELISA inhibition assays in the SCP studies, two subjects (2 and 43) were chosen that recognized rSCP with high intensity by immunoblot analysis (FIG. 5B). Two subjects were compared, one with high IgE to shrimp (52 kU/L) and another with a low level (7 kU/L), to gauge the relative importance of SCP as an allergen, relative to tropomyosin (Lit v 1) on an individual basis. Serum was incubated with increasing concentrations of rSCP, rLit v1, and BS extract, and rCor a 9 as a negative control. rSCP, rLit v 1, and BS were each able to inhibit the binding of the subjects' IgE antibodies to the solid phase-coated BS, whereas the negative control showed no inhibition (FIGS. 8A and 8B). Maximal inhibition was obtained with rSCP at 25 mg/mL (79%) compared with 27% with rLit v 1 for subject 43 (FIG. 8B), and 48% compared to 44%, respectively, for subject 2, whereas 100% inhibition was seen with BS extract (FIG. 8A).

### EXAMPLE 8

rSCP Causes Mediator Release from a RBL-2H3 Cell Line

The SCP studies also involved an investigation of rSCP induced mediator release from rat basophilic leukemia cells. RBL cells passively sensitized with shrimp-specific IgE antibodies from a pool containing sera from 3 subjects (14, 47, and 48) with high levels of shrimp-specific IgE (100 kU/L, 100 kU/L, 80 kU/L), recognizing rSCP with high intensity by immunoblot, were used to compare mediator release induced by rSCP, rLit v 1, and BS extract. The highest mediator release was obtained with BS extract (40%). Mediator release induced by rSCP was significant, approaching 30% of the total possible release (i.e., release induced by Triton X), or 65% of the release obtained with BS extract. Interestingly, release induced by rSCP was higher than that induced by rLit v 1 (20% of total possible release, or 44% of the release induced by BS) for this particular pool of patients' sera (FIG. 8C).

Crustaceans are responsible for food-induced allergic reactions in both children and adults. In spite of the high prevalence of crustacean allergy, there is limited information regarding the proteins involved in the induction of such allergic reactions. Until very recently, tropomyosin was the only major shrimp allergen identified. A minor shrimp allergen, arginine kinase (Pen m 2, Lit v 2; SEQ ID NO: 58), has been described in raw shrimp. In the present disclosure, myosin light chain (MLC) and sarcoplasmic calcium-binding protein have been isolated, characterized and identified as major shrimp allergens.

MLC protein (Lit v 3; SEQ ID NO: 59), disclosed herein as a shrimp allergen, was recognized by more than 50% of the subjects with shrimp allergy. Although tropomyosin appears to be the most abundant allergen in crustaceans, some of the examined patients showed predominant binding to Lit v 3. In two patients, Lit v 3 was almost the only allergen recognized. Therefore, it is important to include Lit v 3 in future diagnostic and therapeutic strategies.

Myosins are a large superfamily of motor proteins that move along actin filaments while hydrolyzing adenosine triphosphate. In muscle, myosin is composed of 2 heavy chains. The globular motor domains interact with actin, whereas the tails dimerize in a coiled-coil structure. Two light chains, each of 20 kD, wrap around the neck region of each myosin heavy chain. While resting, troponin prevents actin-myosin interaction. During sarcolemmal depolarization, cytosolic calcium increases and binds troponin C, which undergoes conformational change, moving the attached tropomyosin away from the myosin-binding sites on actin filaments. The actin-myosin complex is influenced by phosphorylation of MLC, which leads to myosin conformational change and translocation of attached thin filaments, causing muscle contraction.

Many shrimp allergens are abundant in shrimp muscle. Tropomyosin (SEQ ID NO: 61) is the best characterized, and its IgE binding epitopes have been identified. Arginine kinase, with a molecular weight of 40 kD, is also found in muscle in significant quantities. The MLC (SEQ ID NO: 59) and SCP (SEQ ID NO: 60) shrimp allergens disclosed herein are also muscle proteins. Exemplary epitopes for antigenic shellfish polypeptides are provided as features of the protein sequences for MLC (SEQ ID NO: 59), SCP (SEQ ID NO: 60), Arginine Kinase (SEQ ID NO: 58), and Tropomyosin (SEQ ID NO: 61). For each of these epitope domains, moreover, it is contemplated that smaller peptides of no fewer than six amino acids that contain a part of the sequence of a given epitope domain will function as epitopes. In addition, the sequences associated with each allergenic protein are exemplary sequences. The disclosure contemplates slightly variant sequences, such as amino acid sequences that vary at 1, 2 or 5 positions (i.e., 1, 2 or 5 residues), in a given species of shellfish, and sequence identities of 90%, 95%, 99% and 99.5% for orthologous proteins, or their coding regions, across species of shellfish.

MLC is recognized in both raw and boiled shrimp extracts, emphasizing the fact that the concentration of an allergen in foods, and its stability to heat and other factors during processing, are important factors in determining the allergenicity of the proteins. MLC or Lit v 3 has 177 amino acids, a molecular weight of 20 kD, and a calculated isoelectric point of 4.2. Lit v 3 and SCP appear to have similar molecular weights and isoelectric points. It is therefore difficult to determine which protein is recognized by a particular patient's IgE antibodies by using standard laboratory methods. In the studies disclosed herein, however, we have demonstrated the allergenicity of recombinant MLC, which was recognized by 17 of 19 subjects tested. This demonstrated the utility of using recombinant allergens to correctly identify allergenic proteins and to appropriately profile allergenic proteins to which an individual patient is reactive.

Identification of particular allergenic proteins with recombinant allergens in a patient is of interest because it would allow the customized design of immunotherapy a la carte or personalized immunotherapy. Recombinant allergens are therefore important tools for the diagnosis of shellfish allergy and for use as immunotherapeutic agents.

IgE binding to a 20-kD protein is detected in both raw and boiled shrimp extracts, sometimes with stronger IgE-binding intensity to the raw protein extract. This is in contrast to several other shrimp proteins that appear to have stronger IgE recognition to boiled extracts. IgE binding to the boiled form of MLC appears to be greater in adults compared to children (see patients 2-6), who tend to recognize the MLC in the raw extract with higher intensity. Interestingly, some children (patients 2, 5, and 6) had asthmatic episodes when exposed to the steam of boiling shrimp, indicating that MLC is aerosolized and might contribute to respiratory symptoms in such patients. Furthermore, protein profiles of boiling water, steam, and boiled shrimp were very similar, consistent with some proteins becoming aerosolized during boiling.

Crustacean proteins are highly cross-reactive and, usually, avoidance of all crustaceans is recommended. Tropomyosin has been identified as the main cross-reactive molecule among crustaceans. Important in vitro cross-reactivity exists with mollusks and other invertebrates, such as dust mites and cockroach. The cross-reactivity among crustaceans, cockroach, and dust mites seems to be based on sequence similarities of tropomyosin IgE-binding epitopes. Also, arginine kinase has been described as a cross-reacting allergen among crustaceans and between crustaceans and insects. Interestingly, the amino acid sequence of MLC is 66% similar and 51% identical to cockroach Bla g 8, the allergenic MLC of B. germanica. Sequence similarity between MLCs can be implicated in in vitro and, possibly, in clinical cross-reactivity among shrimp and cockroach and, possibly, dust mites. In contrast, sequence identity with other invertebrate MLCs, such as Schistosoma (13% identity) and Aedes (17% identity) species, was low. Although sensitization to D. pteronyssinus is detected in 41% of the Spanish asthmatic population (Madrid, 12%; Canary Islands, 72%), the house dust mite sensitization rate among subjects with shellfish allergy was much higher: 37 (97%) of 38 of the patients examined by the inventors (90% of the Madrid patients and 100% of the Canary Island patients) were sensitized to dust mites. Also, 89% of our subjects were sensitized to cockroach (100% of the patients in Madrid, 80.9% of the patients in the Canary Islands, and 100% of the patients in New York). Although not all subjects with dust mite or cockroach allergy have crustacean allergy, it is expected that sensitization to particular dust mite or cockroach allergens, possibly tropomyosin, MLC, or others, will contribute to the induction of allergy to crustaceans. To date, however, the initial sensitizing protein remains unknown. Additionally, crustacean muscle proteins are particularly allergenic, but homologous vertebrate proteins are not allergens. Although many of those proteins are phylogenetically conserved throughout evolution, substantial amino acid substitutions appear to abolish IgE binding to vertebrate proteins, as has been shown for tropomyosin. This is of importance in the design of mutated hypoallergenic molecules for use in the treatment of individuals with shellfish allergy and can be applied to the design of hypoallergenic tropomyo sin and MLC variants.

In this disclosure, SCP was also characterized as a new shrimp allergen, i.e., Lit v 4.0101. Some subjects who recognized a 20-kd shellfish (shrimp) protein by IgE immunoblotting showed little binding to MLC or rLit v3, indicating the presence of another allergenic protein of similar molecular weight. Subsequent LC-MS/MS analysis of a 20-kd protein from a 2-dimensional gel yielded the sequence of multiple peptides identified as belonging to a SCP. rSCP was recognized by the IgE of subjects with shrimp allergy. Lit v 4.0101 has 194 amino acids, a molecular weight of 22 kd, and a calculated isoelectric point of 4.7. High sequence identity with α-B and α-A chains (93.8%) from Penaeus spp (P02636) and 80% with the β chain (P02635) indicate that the cloned allergen is a member of the α chain family. SCPs are acidic cytosolic EF-hand type Ca²⁺ binding proteins (20-22 kd). In shrimp, SCPs are dimers of two polypeptide chains (αα, αβ, and ββ), with 3 calcium-binding sites in each chain. The aligned amino acid sequences of different SCP molecules are shown in FIG. 6.

Although the precise function of muscle SCP has not been determined, it has been speculated that invertebrate SCP may serve a function similar to the function of vertebrate parvalbumins—that is, promoting rapid muscle relaxation by facilitating calcium translocation from myofibrils to the sarcoplasmic reticulum—and may protect against high calcium concentration inside the cell. The amino acid composition and physicochemical characteristics of different SCPs suggest that they are not conserved proteins. Because the biological function of SCP may be carried out without interacting with other proteins, there is little need to conserve surface amino acid residues. Sequence identity between shrimp and scallop SCP (P02637), for instance, is only 14%, and 18% to 52% with Drosophila (NP_{—}001015389 and NP_{—}524381;NP_{—}524381) (FIG. 6). This is consistent with the lack of in vitro cross-reactivity seen by immunoblot with cockroach, dust mite, and mollusk SCPs. Although most of the 52 subjects are also sensitized to dust mite and cockroach, SCP does not appear involved in cross-reactivity among crustaceans and other arthropods. In contrast, high sequence identity with crawfish SCP (81% to 82%; ABB58783, P05946; FIG. 6) helps explain cross-reactivity among crustacean SCPs, as was shown by immunoblot inhibition with lobster and crab extracts. In summary, although sensitization to tropomyosin has been implicated in cross-reactivity between crustaceans and mollusks, and also with other arthropods, sensitization to SCP appears to be involved only in cross-reactivity among crustaceans. Similar to previously identified shrimp allergens, SCP is also a muscle protein. Interestingly, parvalbumin, troponin C, MLC, and SCP are all EF-hand-type Ca²⁺ binding proteins. EF-hand-type proteins with a variable number of EF motifs are allergenic proteins found in tree pollens (Bet v 4, Ole e 3, Ole e 8), grass pollens (Ph1 p7), rapeseed (Bra n 1, Bra n 2), and some vertebrates such as fish (Gad m 1, Sal s 1) and frog (Ran e 1, Ran e 2). Parvalbumins are important fish allergens. Also among invertebrates, troponin C is a minor cockroach allergen (Bla g 6). Although amino acid sequence identity of shrimp SCP with other EF-hand-type Ca²⁺ binding proteins is low (12% sequence identity with cod parvalbumin Gadm1, cockroach troponin C Bla g 6, cockroach MLC Bla g 8, and shrimp MLC Lit v 3), it has been suggested that all are derived from a common ancestral protein because they possess the common structure of Ca²⁺-binding sites. Although sequence identity cannot explain their common allergenicity, repetitive structures (calcium binding sites) may act as allergenic epitopes in all these proteins. This has been shown for cod parvalbumin, in which modification of these calcium binding sites by calcium depletion or mutagenesis can decrease IgE binding. However, inhibition studies among different EF-hand proteins from pollen and vertebrates have shown limited cross-reactivity, suggesting that different families of calcium-binding allergens may possess specific epitopes involved in cross-reactivity within members of the same family. The data disclosed herein also support this lack of cross-reactivity among EF-hand proteins, which appears in general limited to phylogenetically closely related species, within one protein type—that is, only among SCPs of crustaceans, but not with MLC, troponin, or other EF proteins of arthropods.

Although tropomyosin is the most abundant allergen in crustaceans, some of the subjects primarily recognized SCP. rSCP was recognized by 38% (20/52) of our subjects with shrimp allergy. Interestingly, 17 of 23 (74%) of children recognized rSCP compared with 3 of 29 (10%) adults, indicating that SCP is an important allergen in the pediatric population. ELISA inhibition experiments showed that a significant proportion of some subjects' shrimp-specific IgE (as much as 78%) is inhibited by rSCP, demonstrating that for some subjects, SCP may be more important than tropomyosin as a shellfish allergen (FIG. 8). Furthermore, the functional RBL-based mediator release assay confirmed that for a subset of subjects (because it has been tested only with a limited number), SCP appears to be a more potent basophil activator than tropomyosin. β-Hexosaminidase release induced by rSCP reached 30% of total maximal release, whereas release induced by recombinant tropomyosin was under 20%. Therefore, SCP should be included in future diagnostic and therapeutic strategies, particularly when children are involved. In summary, we have identified shrimp SCP as a new shrimp allergen named Lit v 4.0101 that is of major importance, particularly in children. SCP is recognized by 38% of the subjects examined and, for some, SCP is the main shrimp allergen recognized. Because Lit v 4.0101 is the predominant shrimp allergen recognized by some subjects, inclusion of SCP in the design of mutated hypoallergenic variants for use in future vaccines for individuals with shellfish allergy is indicated and is contemplated.

### EXAMPLE 9

Shrimp Sensitization Decreases with Age

In an effort to identify the IgE-binding epitopes of shrimp allergens and to characterize epitope recognition profiles of children and adults with shrimp allergy, 53 subjects, 34 children and 19 adults, were selected with immediate allergic reactions to shrimp, increased shrimp-specific serum IgE levels, and positive immunoblot binding to shrimp. Study subjects and 7 nonatopic control subjects were tested by means of peptide microarray for IgE binding with synthetic overlapping peptides spanning the sequences of Litopenaeus vannamei shrimp tropomyosin (Lit v 1), arginine kinase (AK) (Lit v 2), myosin light chain (MLC) (Lit v 3), and sarcoplasmic calcium-binding protein (SCP) (Lit v 4). The Wilcoxon test was used to determine significant differences in z scores between patients and control subjects.

A library of overlapping peptides consisting of 15 amino acids with an offset of 3 corresponding to the primary sequence of the 4 allergans found in pacific white shrimp Litopenaeus vannamei tropomyosin (Lit v 1), arginine kinase (AK) (Lit v 2), myosin light chain (MLC) (Lit v 3), and sarcoplasmic calcium-binding protein (SCP) (Lit v 4) was commercially synthesized by using the PepStar technique (JPT Peptide Technologies, Berlin, Germany). Peptides were diluted (1:2) with Protein Printing Buffer (ArrayIt Corp., Sunnyvale, Calif.) and printed in two sets of duplicates onto SuperEpoxy glass slides (ArrayIt Corp) by using the NanoPrint Microarrayer 60 (ArrayIt Corp.). In addition, printed arrays included Protein Printing Buffer alone as negative control spots for background normalization, and fluorochrome-labeled BSA as a reference for grid alignment (positioned grid controls). After printing, the slides were dried overnight at room temperature before use.

The median shrimp IgE level was 4-fold higher in children than in adults (47 vs 12.5 kU(A)/L). The frequency of allergen recognition was higher in children (tropomyosin, 81% [94% for children and 61% for adults]; MLC, 57% [70% for children and 31% for adults]; AK, 51% [67% for children and 21% for adults]; and SCP, 45% [59% for children and 21% for adults]), whereas control subjects showed negligible binding. Seven IgE-binding regions were identified in tropomyosin (see Table III(A)) by means of peptide microarray, confirming previously identified shrimp epitopes. In addition, 3 new epitopes were identified in tropomyosin (epitopes 1, 3, and 5b-c); 5 epitopes were identified in MLC (see Table III(B)); 3 epitopes were identified in SCP (see Table III(C)); and 7 epitopes were identified in AK (see Table III(A)). An IgE-binding epitope was defined as containing at least two contiguous peptides. In some cases, more than four or five continuous peptides were bound by patients' IgE. Because they are likely to contain more than one epitope, those peptides are said to form an IgE binding region. For example, tropomysoin epitopes 5a, 5b, and 5c form an IgE-binding region. The frequency of recognition of each peptide represents the number of patients with an average weighted z score of greater than 3 for that particular peptide divided by the total number of patients who recognize that allergen. Peptides are considered to represent an epitiope when the average weighted z score is greater than 3 (p<0.003) or 2 for AK (p<0.05) and are recognized by at least 20% of the subjects who recognize that particular protein.

Immunolabeling was carried out as previous described by Lin et al. (J. Allergy Clin. Immunol. 124: 315-22, 2009) with some modifications. In brief, the slides were blocked with 400 μl of 1% human serum albumin (HAS) in PBS containing Tween 20 (0.05%) (PBS-T) for 60 minutes at RT, followed by incubation with 250 μl of the patient's serum diluted 1:5 in PBS-T/HAS for 24 hours at 4° C. Slides were then washed with PBS-T and incubated for 24 hours at 4° C. with a mix of 3 monoclonal biotinylated anti-human IgE antibodies, one from Invitrogen (Carlsbad, Calif.), one from BD Biosceinces PharMingen (San Jose, Calif.), and one as a gift from Phadia (Uppsala, Sweden) that was biotinylated and diluted 1:1000 in PBS-T/HAS. Slides were then incubated for 3 hours at 31° C. with Anti-Biotin-Dendrimer Oyster 550 (350; Genisphere, Hatfield, Pa.) in Dendrimer Buffer (Genisphere) at 0.6 μg/mL with the addition of 0.02 μg/mL salmon sperm DNA (Invitrogen), followed by washing with PBS-T, 15 mmol/L Tris, 0.1×PBS, and 0.05×PBS. Slides were subject centrifugation and dried with a Scan Array Gx (PerkinElmer, Waltham, Mass.). Images were saved in TIF format.

The fluorescence signal of each spot was digitized with the program ScanArray Express (PerkinElmer), exported as comma-delimited text files, and transformed to z scores as described by Lin et al. (J. Allergy Clin. Immunol. 124: 315-22, 2009). An index z value of each peptide element was generated from the median of z scores of the 4 replicates. I an individual peptide sample had a z score exceeding 3 or 2, it was considered positive, indicating that the signal was above the background with a P value of less than 0.003 or 0.05, respectively.

IgE-binding epitopes are defined as including 2 or more overlapping peptides, and therefore subsequent analysis was carried out by adjusting each z score to the median of itself and the 2 flanking peptides (weighted average z score), by using the following formula: z=25*_{z-1}+0.5*Z₀+0.25*Z₊₁. An IgE-binding epitope for the population analyzed contained at least 2 contiguous peptides with a weighted average z score of greater than 3 for tropomyosin, MLC, and SCP or greater than 2 for AK. A lower z score was considered for AK for epitope identification because of the lower intensity of binding of the AK peptides. The Wilcoxon test and q values (calculated by the R project (Storey et al., J. R. Stat. Soc. B. 66: 187-205, 2004)) were used to determine statistical differences between weighted average z scores of atopic and negative control subjects and also between children and adults. A P value of less than 0.05 and a false discovery rate (FDR) of 0.05 were selected as significant thresholds. FDR and q values were used to adjust for the multiple comparisons in the peptide microarray study.

Inhibition experiments were carried out with selected peptides as inhibitor to demonstrate that the fluorescent signal from the peptide spots was specific (IgE mediated). For each protein, 1 or 2 peptides were selected that were bound by the subject's Ige antibodies and were included within an IgE-binding epitope. A serum pool was preincubated with individual peptides for 1 hour, followed by incubation of the serum/peptide mixture with the printed microarray slide. As expected, peptides inhibited IgE binding to the identical sequence on the printed, as well as peptides that were immediately adjacent. This was expected because each peptide overlaps with adjacent peptides by 12 of 15 amino acids. Interestingly, in the case of tropomyosin, a 15 amino acid peptide in epitope 2 in Table III (epitope 2: amino acids 43-57 of SEQ ID NO: 60) inhibited not only the same sequence included in peptides 13 to 15 (epitope 2: amino acids amino acids 43-63 of SEQ ID NO: 60), but also peptides 2 to 3 (epitope 1: amino acids 4-21 of SEQ ID NO: 60), peptides 29 to 31 (epitope 4: amino acids 85-105 of SEQ ID NO: 60), and peptides 84 to 85 (epitope 7: amino acid 246-264). Sequence analysis of all of these areas showed a common motif, LEX₁X₂L (SEQ ID NO: 62) or LEX₁X₂N (SEQ ID NO: 63) where X₁ can be D, E, N, or K, and X₂ can be D or E.

In a particular aspect, IgE epitopes identified in tropomyosin, MLC, SCP, and AK with their frequency of recognition are set out in Table III. For each of these epitope domains, it is contemplated that smaller peptides of no fewer than six amino acids that contain a part of the sequence of a given epitope domain will function as epitopes. In addition, the sequences associated with each allergenic protein are exemplary sequences. The disclosure contemplates slightly variant sequences, such as amino acid sequences that vary at 1, 2 or 5 positions (i.e., 1, 2 or 5 residues), in a given species of shellfish, and sequence identities of 90%, 95%, 99% and 99.5% for orthologous proteins, or their coding regions, across species of shellfish.

TABLE III
IgE Epitopes and Frequency
A. Tropomyosin (SEQ ID NO: 61)

| | Epitope | | | | | | | | |
|---|---|---|---|---|---|---|---|---|---|
| | 1 | 2 | 3 | 4 | 5a | 5b | 5c | 6 | 7 |
| Frequency | 65 | 60 | 35 | 30 | 50 | 55 | 60 | 35 | 50 |
| (%) | | | | | | | | | |
| AA | 1-36 | 37-63 | 61-81 | 82-105 | 115-150 | 142-162 | 157-183 | 190-210 | 246-284 |

B. MLC (SEQ ID NO: 59)

Epitope

| | 1 | 2 | 3 | 4a | 4b | 5 |
|---|---|---|---|---|---|---|
| Frequency | 45 | 50 | 30 | 70 | 35 | 75 |
| (%) | | | | | | |
| AA | 13-30 | 22-48 | 49-66 | 58-90 | 79-99 | 118-141 |

C. SCP (SEQ ID NO: 60)

| | Epitope | | |
|---|---|---|---|
| | 1 | 2 | 3 |
| Frequency | 80 | 40 | 25 |
| (%) | | | |
| AA | 10-36 | 49-72 | 130-147 |

D. AK (SEQ ID NO: 58)

| | Epitope | | | | | | | |
|---|---|---|---|---|---|---|---|---|
| | 1 | 2 | 3 | 4a | 4b | 5 | 6 | 7 |
| Frequency | 45 | 25 | 50 | 40 | 30 | 60 | 30 | 70 |
| (%) | | | | | | | | |
| AA | 1-18 | 25-42 | 64-96 | 121-141 | 142-159 | 160-192 | 232-255 | 319-342 |

Interestingly, frequency of individual epitope recognition, as well as intensity of IgE binding, was significantly greater in children than in adults for all four proteins. Children with shrimp allergy have greater shrimp-specific IgE antibody levels and show more intense binding to shrimp peptides and greater epitope diversity than adults.

Numerous modifications and variations of the technology disclosed herein are possible in view of the above teachings and are within the scope of the invention. The entire disclosures of all publications cited herein are hereby incorporated by reference.

1. A method of treating a shellfish allergic reaction comprising administering a therapeutically effective amount of a specific binding partner of a shellfish protein selected from the group consisting of myosin light chain, sarcoplasmic calcium-binding protein, fatty acid binding protein, hemocyanin and troponin C.

2. The method according to claim 1 wherein the shellfish protein is derived from shrimp, prawn, crab, lobster or crawfish.

3. The method according to claim 2 wherein the shellfish protein is derived from shrimp selected from the group consisting of black tiger shrimp and white shrimp.

4. The method according to claim 1 wherein the specific binding partner is an antibody or antibody fragment.

5. The method according to claim 4 wherein the antibody or antibody fragment is a monoclonal antibody or fragment thereof.

6. The method according to claim 1 further comprising administering a therapeutically effective amount of a second binding partner specifically recognizing a second shellfish protein selected from the group consisting of myosin light chain, sarcoplasmic calcium binding protein, tropomyosin, arginine kinase, hemocyanin, fatty acid binding protein and troponin C, wherein the first and second shellfish proteins are different.

7. The method according to claim 6 wherein the second shellfish protein is derived from shrimp, prawn, crab, lobster or crawfish.

8. The method according to claim 7 wherein the second shellfish protein is derived from shrimp selected from the group consisting of black tiger shrimp and white shrimp.

9. The method according to claim 6 wherein the specific binding partner is an antibody or antibody fragment.

10. The method according to claim 9 wherein the antibody or antibody fragment is a monoclonal antibody or fragment thereof.

11. A method of ameliorating or preventing a shellfish allergic reaction comprising administering a therapeutically effective amount of a protein selected from the group consisting of shellfish myosin light chain, shellfish sarcoplasmic calcium-binding protein, shellfish fatty acid binding protein, shellfish hemocyanin, shellfish troponin C and a specific binding partner thereof.

12. The method according to claim 11 wherein the shellfish protein is derived from shrimp, prawn, crab, lobster or crawfish.

13. The method according to claim 12 wherein the shellfish protein is derived from shrimp selected from the group consisting of black tiger shrimp and white shrimp.

14. The method according to claim 11 wherein the specific binding partner is an antibody or antibody fragment.

15. The method according to claim 14 wherein the antibody or antibody fragment is a monoclonal antibody or fragment thereof.

16. The method according to claim 11 further comprising administering a therapeutically effective amount of a second protein selected from the group consisting of shellfish myosin light chain, shellfish sarcoplasmic calcium binding protein, shellfish tropomyosin, shellfish arginine kinase, shellfish hemocyanin, shellfish fatty acid binding protein, shellfish troponin C and a specific binding partner thereof, wherein the first and second proteins are different.

17. The method according to claim 16 wherein the second protein is derived from shrimp, prawn, crab, lobster or crawfish.

18. The method according to claim 17 wherein the second protein is derived from shrimp selected from the group consisting of black tiger shrimp and white shrimp.

19. The method according to claim 16 wherein the specific binding partner is an antibody or antibody fragment.

20. The method according to claim 19 wherein the antibody or antibody fragment is a monoclonal antibody or fragment thereof.

21. A method of diagnosing a risk of an allergic reaction to shellfish comprising contacting an immunoglobulin-containing biological sample of a subject with a shellfish protein selected from the group consisting of myosin light chain and sarcoplasmic calcium-binding protein and measuring a reaction between the sample and the protein, wherein a reaction leads to a diagnosis of a subject at risk of a shellfish allergic reaction.

22. The method according to claim 21 wherein the shellfish protein is derived from shrimp, prawn, crab, lobster or crawfish.

23. The method according to claim 22 wherein the shellfish protein is selected from the group consisting of black tiger shrimp and white shrimp.

24. The method according to claim 21 further comprising contacting the biological sample with a second shellfish protein selected from the group consisting of myosin light chain, sarcoplasmic calcium-binding protein, tropomyosin, arginine kinase, hemocyanin, fatty acid binding protein and troponin C and measuring a reaction between the sample and the protein, wherein the first and second shellfish proteins are different and wherein a reaction leads to a diagnosis of a subject at risk of a shellfish allergic reaction.

25. The method according to claim 24 wherein the second shellfish protein is derived from shrimp, prawn, crab, lobster or crawfish.

26. The method according to claim 25 wherein the second shellfish protein is selected from the group consisting of black tiger shrimp and white shrimp.

27. The method according to claim 20 wherein the monoclonal antibody or fragment thereof binds to an epitope comprising an amino acid at any one of amino acid positions 1-18, 25-42, 64-96, 121-141, 142-159, 160-192, 232-255, or 319-342 in the amino acid sequence of SEQ ID NO: 58.

28. The method according to claim 20 wherein the monoclonal antibody or fragment thereof binds to an epitope comprising an amino acid at any one of amino acid positions 13-30, 22-48, 49-66, 58-90, 79-99, or 118-141 in the amino acid sequence of SEQ ID NO: 59.

29. The method according to claim 20 wherein the monoclonal antibody or fragment thereof binds to an epitope comprising an amino acid at any one of amino acid positions 10-36, 49-72, or 130-147 in the amino acid sequence of SEQ ID NO: 60.

30. The method according to claim 20 wherein the monoclonal antibody or fragment thereof binds to an epitope comprising an amino acid at any one of amino acid positions 1-36, 37-63, 61-81, 82-105, 115-150, 142-162, 157-183, 190-210, or 246-284 in the amino acid sequence of SEQ ID NO: 61.

31. The method according to claim 20 wherein the monoclonal antibody is a recombinant antibody.

32. The method according to claim 31 wherein the recombinant antibody is selected from the group consisting of a human chimeric antibody, a humanized antibody, and a human antibody.

33-58. (canceled)


SEQUENCE LISTING (69 sequences)

SEQ ID NO: 1 - protein, 13 aa, Litopenaeus vannamei
  misc_feature (4)..(4): Xaa can be any naturally occurring amino acid
KGGXNVFDMFTQK

SEQ ID NO: 2 - protein, 16 aa, Litopenaeus vannamei
SSGESDDDDVVAASIR

SEQ ID NO: 3 - DNA, 1135 bp, Litopenaeus vannamei
gtgtgatcagcgccgcagacagacagccatgtcccgcaagtcaggctctcgctcctcctccaagaggtccaagaaatccggaggaggaagcaatgtgttcgacatgttcacgcagcggcaggtggccgagttcaaggagggcttccagctgatggaccgcgacaaggacggcgtcatcggcaagaccgacctgcgcggaaccttcgacgagatcggccgcatcgccacggaccaggagctcgacgagatgctggccgacgcgcccgcgcccatcaacttcaccatgttgctcaacatgttcgcggaacggcagacgggcgagagtgacgacgacgacgtggtggcgaaggccttcctggcgttcgccgacgaggaaggcaacatcgactgcgacaccttccgccacgccctcatgacctggggcgacaagttctcctcgcaggaagccgacgacgccctcgaccagatggacatcgacgacggaggcaagatcgacgtccaaggcgtcatccagatgctcacagccggcggcggagacgacgcggccgccgaggaagcctaaagtggaagtccttttgaaagtccttgagatcctttctcacccctctgacccctcctccgagtccttccgtcctcaccccccaacccctaccccatttcctcggaaactctcttcccttcacacgcaaggcacgcacccacacacacgccctcacgcacgctcacgccttcgtccgcacgttcctggtccccccttcacaagcccgcccttccgcccaccctcgttgtccgtcactgccagtggggcgtgctgcgtcctccctccccccgcccccgcccccgcccgccctcctccctcctgctccctcccttctccttctggtgctgtgactgttgccggcgaagtgatcgctgctcggcgacgacccggggaaaaggaacggaaaatgaatgaatatggagagaagaaacaggagagagcgaggaaatgcgaaatggagagataccaaacaaagaaaaggaaaaagacaagcgataaacgaaggcatattctatagtgatctacgatatatttattgttgaaattaatcgtacttaaataaagtataagttggataaaaaaaaaaaaaaaaaa

SEQ ID NO: 4 - protein, 177 aa, Litopenaeus vannamei
MSRKSGSRSSSKRSKKSGGGSNVFDMFTQRQVAEFKEGFQLMDRDKDGVIGKTDLRGTFDEIGRIATDQELDEMLADAPAPINFTMLLNMFAERQTGESDDDDVVAKAFLAFADEEGNIDCDTFRHALMTWGDKFSSQEADDALDQMDIDDGGKIDVQGVIQMLTAGGGDDAAAEEA

SEQ ID NO: 5 - DNA, 582 bp, Litopenaeus vannamei
atggcttacagctgggacaaccgcgtcaagtacgtcgtcaggtacatgtacgacattgacaacaatggcttcttggacaagaacgacttcgagtgccttgccgtcaggaacaccctgattgagggccgtggcgaattcagcgccgatgcctatgccaacaaccagaagatcatgaggaacctctggaacgagatcgccgagctcgctgacttcaacaaggacggcgaggtaactgttgacgagttcaagcaggctgtgcagaagcattgccagggcaagaaatacggcgatttccccggtgccttcaaggttttcattgccaaccagttcaaggctattgacgttaacggcgacggcaaggttggcctcgatgagtacaggcttgactgcattaccaggtctgcctttgctgaggtgaaggagattgatgacgcttacaacaagctcaccactgaggacgacaggaaggccggcggactcaccctggagcgctaccaggacctgtacgctcagttcatctccaaccccgatgagtcttgcagcgcctgctaccttttcggacccctgaaggtggtgcagtag

SEQ ID NO: 6 - protein, 193 aa, Litopenaeus vannamei
MAYSWDNRVKYVVRYMYDIDNNGFLDKNDFECLAVRNTLIEGRGEFSADAYANNQKIMRNLWNEIAELADFNKDGEVTVDEFKQAVQKHCQGKKYGDFPGAFKVFIANQFKAIDVNGDGKVGLDEYRLDCITRSAFAEVKEIDDAYNKLTTEDDRKAGGLTLERYQDLYAQFISNPDESCSACYLFGPLKVVQ

SEQ ID NO: 7 - DNA, 411 bp, Litopenaeus vannamei
  misc_feature: Fatty acid binding-protein (FABP) coding region
atggctaagattgaaggaaagttcaagatggagagctccgagaactttgacgagttcatgaaggctctgggtgttggcttggtgatgcgcaagatgggtaacgctgcgacccccactgtcgagatcaccaaggacggcgatacctacacaatgaagacgaccaccactttcaagaccacagagatcaagttcaagttgggggaggaatttgaagagaccacagctgatggccgcgttgtaaagtcaaccattactttggatggcaataagctagttcacaagcaagttggagacaaggagaagaaagagaaggattctgagcttctccgagaattcaccgacgacaagatgctaatggagtgcaaggttgatgacgttgtctgcaagcgagtttactctcgtttagaataa

SEQ ID NO: 8 - protein, 136 aa, Litopenaeus vannamei
  MISC_FEATURE: Fatty acid binding-protein (FABP)
MAKIEGKFKMESSENFDEFMKALGVGLVMRKMGNAATPTVEITKDGDTYTMKTTTTFKTTEIKFKLGEEFEETTADGRVVKSTITLDGNKLVHKQVGDKEKKEKDSELLREFTDDKMLMECKVDDVVCKRVYSRLE

SEQ ID NO: 9 - DNA, 2192 bp, Marsupenaeus japonicus
gaccgttaacatgaaggtcttggtgctgttcgctctcgttgcggctgctgccgcctggccgagctttggcttccagtcggacgccggaggagtgtctgacgcccagaagcaacatgacatcaacttcctgctgcacaagatctacggggatatccgtgatgatgccctgaaggccaaggctgattcctttgacccggaggctgatctatcccattacagtgacgacggtgaagcagtgcatacactcatcagagacctcaaggatgacaggcttctccaacagaagcactggttctctctcttcaactccaggcagcgtcatgaagcacttatgctattcgatgtcctcattcattgcaaggactgggatacattcgtcagcaatgctgcctacttccgccaacgtatgaatgagggagagtttgtcaatgccttgtatgttgcggtcatccactcatccttggctgagtatgttgtacttccacctctgtatgaggttacccctcacctctttaccaacagtgaggttatcgaggcagcttatcgtgctaagcagacacaaaagccaggtaaattcaagtcgtcctttactggaactaagaagaaccctgaacaaagagttgcctactttggagaggacattggaatgaacactcaccacgtgacttggcatatggaattcccattctggtggcaggacaaatacagccatcatctggatcgcaaaggagagaacttcttctgggttcatcatcaacttaccgtccgttttgatgccgaacgtctttccaattacttggatcctgtagatgagcttcactgggaaaagcccatcgtacaaggctttgctccccacaccacttacaagtatggaggtcagttcccctctcgtccagataatgcacgattcgaggatgtggacggcgttgcccgaattcgagacttgctcattgttgagagccgaatccgtgatgctattgcccacggttacattgtcgacagggaaggcaaacacattgatatcatgaatgagcgtggtattgacgtccttggagacattatcgaatcgtctttgtacagtcctaatgtccagtactacggggccttgcataatactgctcacattgtgcttggtcgacagagtgatccccatggcaaatacgatttgcccccaggtgtgctagaacacttcgaaactgccacccgtgaccccagtttcttcaggctacataaatatatggataacatcttcaaggaacacaaggacactctccctccttatactgcagaagaactgacatttgctggtgttagtgttgacagtattgcaatcgagggtgcactagaaacgtacttcgaggactttgaatacaatcttattaatgctgttgacgacactgaacaaattccagatgtagaaatctctacatatgtacctcgtctgaatcacaaggactttgctttcaaaattggtgttagcaataacaagggtgaagaaactctggccaccgttcgcattttcgcctggcctcacctcgacaacaatgggattgagttcagcttcgacgagggccgttggcatgctattgaactggataaattctgggttaagttgggcactggagttaccgaaatcactcgcaaatgttcggaatccgcggtgactgttcctgatgtccccagtttcgcaactctctttgagaagaccaaagcagccttgggtggcgccgactccggacttacagatttcgagagtgccactggcattccaaatcgattccttctccccaagggtaacgaaaagggtcttgagttcgaccttgttgtggccgtgaccgatggtgcagctgacgcagcagtggacggacttcacgaaaataccgaattcaaccactatggtgcttatggcaagtaccctgacaaccgcccacatggataccctctggatcgcagcgttccagatgaacgagtattcgaggatcttcctaactttggccacatccaagtaaaggtcttcaatcacggggaacatatccaccatgattaggaaatacctttatgctagataatggtattcttaatagaaatgtggaatatgatgttttgtttcaaggagtgaagatatgtaacttgatataactttatggaatacaaagaaaatgtttccaaaaaaaaaaaaaaaaaaaaaaaaa

SEQ ID NO: 10 - protein, 678 aa, Marsupenaeus japonicus
MKVLVLFALVAAAAAWPSFGFQSDAGGVSDAQKQHDINFLLHKIYGDIRDDALKAKADSFDPEADLSHYSDDGEAVHTLIRDLKDDRLLQQKHWFSLFNSRQRHEALMLFDVLIHCKDWDTFVSNAAYFRQRMNEGEFVNALYVAVIHSSLAEYVVLPPLYEVTPHLFTNSEVIEAAYRAKQTQKPGKFKSSFTGTKKNPEQRVAYFGEDIGMNTHHVTWHMEFPFWWQDKYSHHLDRKGENFFWVHHQLTVRFDAERLSNYLDPVDELHWEKPIVQGFAPHTTYKYGGQFPSRPDNARFEDVDGVARIRDLLIVESRIRDAIAHGYIVDREGKHIDIMNERGIDVLGDIIESSLYSPNVQYYGALHNTAHIVLGRQSDPHGKYDLPPGVLEHFETATRDPSFFRLHKYMDNIFKEHKDTLPPYTAEELTFAGVSVDSIAIEGALETYFEDFEYNLINAVDDTEQIPDVEISTYVPRLNHKDFAFKIGVSNNKGEETLATVRIFAWPHLDNNGIEFSFDEGRWHAIELDKFWVKLGTGVTEITRKCSESAVTVPDVPSFATLFEKTKAALGGADSGLTDFESATGIPNRFLLPKGNEKGLEFDLVVAVTDGAADAAVDGLHENTEFNHYGAYGKYPDNRPHGYPLDRSVPDERVFEDLPNFGHIQVKVFNHGEHIHHD

SEQ ID NO: 11 - DNA, 2120 bp, Marsupenaeus japonicus
gaccatcagcatgaaggtcttagtggttcttgggcttgtggctgcggccgccttccaggtcgttggtgcagatgatgttcagaagcagaaggatatcctgtatcttgtgcacaagatttatggagacatccaggatgctgacctgaaggccactgccaattccttcgaccccgtggcagaccttggcatctacagtgatggtggcgcagccgcccagagactggtcaaggatcttaacgacggaaagctcctacagcagaagcactggttctcccttttcaataccaggcatcgccatgaagcactgctgctttttgatgtcctcattcactgcaacgactgggcaggcttcgtcggaaatgcggcctacttccgccaaaagatgaacgagggagaatttgtttacgccgtgtacgttgccgtcatccactcgcctctggccgaacacgtggtgctccctcccctctacgagatcacaccccacccgttcaccaacagcgaagtcattgaggaagcttatcgtgccaagcagacacagacacccggcaaattcaagtccaccttcacgggaaccaagaaaaaccctgaacaaagagtggcctacttcggtgaggacatcggattgaatacccaccacgttacctggcatatggaattccccttctggtgggacgacaaatacggtcatcacctggatcgcaaaggagagaacttcttctgggttcatcatcaacttaccgtccgcttcgatgccgaacgtctctccaactacttggatccagttggtgaactccagtggcacaaggagatcgtcgaaggctttgctccccacaccacctacaagtatggaggccagttccctactcgtcctgacaacgtcaatttcgaagacgtggacggtgtagctcgaatccgtgacatgaccatcatcgagagccgaattcgcgacgccattgcccatggctacatcgttgacagccacggcaaacatattgacatcaataatgagcgaggcattgacattctgggagacatcatcgagtcatcactgtacagtcccaacgtgcagtactacggagccttacataacactgcccatatcgtgctgggccgtcaagctgatccccatggaaagtacgacttaccccctggtgtgctggaacacttcgaaactgccacccgggaccccagcttcttccggcttcacaagtatatggataacatcttcaaggaacacaaggacacccttaccccctacaccaaggccgatttggaattcgccggcgtctccatcgacaacgtggccgttgagggtgaactggaaacctacttcgaagacttcgaatacagcctcatcaatgcagtcgacgatgctgaaggaattcaggatgtagcgatcagcacttacgtgccccgtctcaaccacaaagaatttaccatcaagcttgatgtaaaatcggatgccgctcgcttggctacagtccgtatctttgcctggcctcacaaggacaacaatggaatcgaatatacatttgatgaaggtcgctggaatgctatcgagctcgataagttctgggtctccctgtcttctggatccaatgccattgagcgcaagtccactgagtctggagtaactgtgccggatgtgccaagcattcaaactctattcgacaaggccgcggcaggcggcgctggcctcaccgaatacgagagtgcaacaggcctacccaacaggttccttctccccaagggcaacgagcaaggtctggagttcgaccttgtggtggccgtgactgatggcgacgccgacgcagctgtagccgatcttcatcagaatactgattacaatcactacggcgcccatggcgtgtaccccgacaagaaacctcacggctatcctctggaccgcaaagttccagatgaacgtgtgttcgaagaactttccaacttcaagcgcatccaagttaaggtcttcaaccatggtgtacacatcgagcacagttaatacctgtttacatctcttcacttgactttgctgatgagtcaattacttccttgaattactcccgataataaagaacatatgcataaaaaaaaaaaaaaaaaaaaaaaaaaaaaaa

SEQ ID NO: 12 - protein, 664 aa, Marsupenaeus japonicus
MKVLVVLGLVAAAAFQVVGADDVQKQKDILYLVHKIYGDIQDADLKATANSFDPVADLGIYSDGGAAAQRLVKDLNDGKLLQQKHWFSLFNTRHRHEALLLFDVLIHCNDWAGFVGNAAYFRQKMNEGEFVYAVYVAVIHSPLAEHVVLPPLYEITPHPFTNSEVIEEAYRAKQTQTPGKFKSTFTGTKKNPEQRVAYFGEDIGLNTHHVTWHMEFPFWWDDKYGHHLDRKGENFFWVHHQLTVRFDAERLSNYLDPVGELQWHKEIVEGFAPHTTYKYGGQFPTRPDNVNFEDVDGVARIRDMTIIESRIRDAIAHGYIVDSHGKHIDINNERGIDILGDIIESSLYSPNVQYYGALHNTAHIVLGRQADPHGKYDLPPGVLEHFETATRDPSFFRLHKYMDNIFKEHKDTLTPYTKADLEFAGVSIDNVAVEGELETYFEDFEYSLINAVDDAEGIQDVAISTYVPRLNHKEFTIKLDVKSDAARLATVRIFAWPHKDNNGIEYTFDEGRWNAIELDKFWVSLSSGSNAIERKSTESGVTVPDVPSIQTLFDKAAAGGAGLTEYESATGLPNRFLLPKGNEQGLEFDLVVAVTDGDADAAVADLHQNTDYNHYGAHGVYPDKKPHGYPLDRKVPDERVFEELSNFKRIQVKVFNHGVHIEHS

SEQ ID NO: 13-14 - skipped (2 entries)

SEQ ID NO: 15 - DNA, 1274 bp, Litopenaeus vannamei
ctcccccaggcaggcccacggtcctagctttctcagttctctttgttggttgagcacctcctaaaaaaaccgccaccatggacgccatcaagaagaagatgcaggcgatgaagctggagaaggataacgccatggacagggcggataccctcgaacagcagaacaaggaggccaacaacagggctgagaagagcgaggaggaggttcacaaccttcagaagaggatgcagcaacttgagaacgaccttgaccaggtgcaggaatccttgctgaaggctaacatccagcttgtggagaaggacaaggccctctctaacgctgagggcgaggtggccgctctgaaccgccgcatccagctgctcgaggaggacctggagcgctctgaggagcgcctcaacaccgccaccaccaagctggccgaggcctcccaggccgccgacgagtccgagcgcatgcgcaaggtgctcgagaaccgctccctgtccgacgaggagcgcatggacgccctggagaaccagctcaaggaggctcgattcctggctgaggaagccgacaggaaatacgacgaggttgcccgtaagctggccatggttgaggccgaccttgagcgtgctgaggagcgtgccgagactggtgaatcaaagatcgtcgagcttgaggaagagctgcgtgtcgttggcaacaacctgaagtcccttgaggtgtctgaggagaaggccaaccagcgcgaggaagcctacaaggagcagattaagacacttaccaacaagctgaaggcggctgaggcccgtgctgagttcgccgagaggtctgtgcagaagctccagaaggaggtcgacaggcttgaagacgaactggttaacgaaaaggagaagtacaagtccattaccgacgagctggaccagactttcagcgaactgtctggctactaaacactctctgctccaaactgttctgccatctctcttcctatgccctcagctggcctgtaacctattttttcataagcttatgtcattcagtttataagaatctttttacttttccttctttttatgtgttgcatttgaccttcgattgagtcaatatcatcactaaatcattcgataacgttggtgaagtctgccatataggtaattttaatgttattaaaaggtaatctttattcaggaacctagattttgaagttcatttcaaaaggacaggtttcatgtattctatggcttaataacataaagaaataaatatttattatcaaaaaaaaaaaaaaaaaa

SEQ ID NO: 16 - protein, 284 aa, Litopenaeus vannamei
MDAIKKKMQAMKLEKDNAMDRADTLEQQNKEANNRAEKSEEEVHNLQKRMQQLENDLDQVQESLLKANIQLVEKDKALSNAEGEVAALNRRIQLLEEDLERSEERLNTATTKLAEASQAADESERMRKVLENRSLSDEERMDALENQLKEARFLAEEADRKYDEVARKLAMVEADLERAEERAETGESKIVELEEELRVVGNNLKSLEVSEEKANQREEAYKEQIKTLTNKLKAAEARAEFAERSVQKLQKEVDRLEDELVNEKEKYKSITDELDQTFSELSGY

SEQ ID NO: 17 - DNA, 480 bp, Litopenaeus vannamei
  misc_feature: Troponin C coding region
  ref <308>: DATABASE ACCESSION NUMBER: GenBank FE066626
  ref <309>: DATABASE ENTRY DATE: 2008-04-01
  ref <313>: RELEVANT RESIDUES: (46)..(525)
ctttcgaaacccggatcgccagtagccatggacagtttggatgaagagcagatcgagaccctccggaaggcgtttgactccttcgacacggagaaaaccggctccatcacagctgagaccatcgccaccatcatgaggatgatgggcgtcaagatctcggagaagaacctgcaggaagccatcgccgagactgacgaggacggatccggcctcctggaattcgaggagttcgtcgagctgtccgccaagttcctcatcgaggaggacgaggaggctctcaaggcggagctcagggaggcttttcgtatttacgataaggaaggaaacggcttcatcaccactgacgtcttgaaggagatcttggccgagcttgaccccaggctgacccccgctgacctcgagaacatcattgaggaggtcgacgaggacggctctggcaccctcgacttcgacgagttcatggagatgatgaacggttaa

SEQ ID NO: 18 - protein, 159 aa, Litopenaeus vannamei
  MISC_FEATURE: Troponin C
LSKPGSPVAMDSLDEEQIETLRKAFDSFDTEKTGSITAETIATIMRMMGVKISEKNLQEAIAETDEDGSGLLEFEEFVELSAKFLIEEDEEALKAELREAFRIYDKEGNGFITTDVLKEILAELDPRLTPADLENIIEEVDEDGSGTLDFDEFMEMMNG

SEQ ID NO: 19 - DNA, 21 bp, Penaeus monodon
tatatgtacgacattgacaac

SEQ ID NO: 20 - DNA, 21 bp, Penaeus monodon
gataagaacgacttcgagtgc

SEQ ID NO: 21 - protein, 7 aa, Penaeus monodon
YMYDIDN

SEQ ID NO: 22 - protein, 7 aa, Penaeus monodon
DKNDFEC

SEQ ID NO: 23 - DNA, 20 bp, Litopenaeus vannamei
ttgaactggttggcaatgaa

SEQ ID NO: 24 - DNA, 22 bp, Litopenaeus vannamei
gtaagcgtcatcaatctcattc

SEQ ID NO: 25 - DNA, 29 bp, Artificial Sequence
  Synthetic nucleotide
aaaaaagcttatggcttacagttgggaca

SEQ ID NO: 26 - DNA, 29 bp, Artificial Sequence
  Synthetic nucleotide
tatttctcgagctgcaccaccttcagggg

SEQ ID NO: 27 - protein, 13 aa, Litopenaeus vannamei
  misc_feature (4)..(4): Xaa can be any naturally occurring amino acid
KGGXNVFDMFTQK

SEQ ID NO: 28 - DNA, 19 bp, Litopenaeus vannamei
  misc_feature (15)..(15): n = inosine
ttygayatgttyacncara

SEQ ID NO: 29 - DNA, 27 bp, Litopenaeus vannamei
aacgtgttcgacatgttcacccagaag

SEQ ID NO: 30 - protein, 13 aa, Litopenaeus vannamei
YMYDIDDDGFLDK

SEQ ID NO: 31 - protein, 22 aa, Litopenaeus vannamei
YMYDIDDDGFLDKNDFECLAVR

SEQ ID NO: 32 - protein, 9 aa, Litopenaeus vannamei
NDFECLAVR

SEQ ID NO: 33 - protein, 13 aa, Litopenaeus vannamei
GEFSAADYANNQK

SEQ ID NO: 34 - protein, 24 aa, Litopenaeus vannamei
NLWNEIAELADFNKDGEVTVDEFK

SEQ ID NO: 35 - protein, 14 aa, Litopenaeus vannamei
NLWNEIAELADFNK

SEQ ID NO: 36 - protein, 10 aa, Litopenaeus vannamei
DGEVTVDEFK

SEQ ID NO: 37 - protein, 17 aa, Litopenaeus vannamei
VFIANQFKAIDVNGDGK

SEQ ID NO: 38 - protein, 8 aa, Litopenaeus vannamei
VFIANQFK

SEQ ID NO: 39 - protein, 9 aa, Litopenaeus vannamei
AIDVNGDGK

SEQ ID NO: 40 - protein, 7 aa, Litopenaeus vannamei
VGLDEYR

SEQ ID NO: 41 - protein, 15 aa, Litopenaeus vannamei
SAFAEVKEIDDAYNK

SEQ ID NO: 42 - protein, 8 aa, Litopenaeus vannamei
EIDDAYDK

SEQ ID NO: 43 - protein, 16 aa, Litopenaeus vannamei
EIDDAYDKLTTEDDRK

SEQ ID NO: 44 - protein, 8 aa, Litopenaeus vannamei
LTTEDDRK

SEQ ID NO: 45 - protein, 9 aa, Litopenaeus vannamei
KAGGLTLER

SEQ ID NO: 46 - protein, 8 aa, Litopenaeus vannamei
AGGLTLER

SEQ ID NO: 47 - protein, 13 aa, Litopenaeus vannamei
YMYDIDNDGFLDK

SEQ ID NO: 48 - protein, 22 aa, Litopenaeus vannamei
YMYDIDNDGFLDKNDFECLAVR

SEQ ID NO: 49 - protein, 9 aa, Litopenaeus vannamei
NDFECLAVR

SEQ ID NO: 50 - protein, 10 aa, Litopenaeus vannamei
DGEVTVDEFK

SEQ ID NO: 51 - protein, 15 aa, Litopenaeus vannamei
DGEVTVDEFKQAVQK

SEQ ID NO: 52 - protein, 8 aa, Litopenaeus vannamei
EIDDAYDK

SEQ ID NO: 53 - protein, 15 aa, Litopenaeus vannamei
YQELYAQFISNEDEK

SEQ ID NO: 54 - DNA, 31 bp, Artificial Sequence
  Synthetic polynucleotide
tttttggatccgatgtcccgcaagtcaggct

SEQ ID NO: 55 - DNA, 31 bp, Artificial Sequence
  Synthetic polynucleotide
tttttctcgagggcttcctctgcggcagcgt

SEQ ID NO: 56 - DNA, 2043 bp, Penaeus vannamei
  misc_feature: Hemocyanin coding region
tggatatctgcagaattcgcccttctcctagccctcgttgcagctgctgccgcctggccgaactttggcttccagtcggacgcaggaggtgagtctgatgcccagaagcagcatgacgtcaacttcctgcttcataagatctacgggaatattcgtgactctgacctaaaagccaaagctgattcctttgaccccgcggccgatttatcccattacagcgatggaggtgaagctgtacagaaacttgttagagaccttaaggatgacaggcttctccaacagaggcactggttctctctcttcaacccgaggcagcgtcatgaagcactcatgcttttcgatgttctcattcactgcaaggactggaatacatttgtcagcaacgctgcctacttccgtcagcatatgaatgaaggagagtttgtctatgctctgtatgttgcagtcatccactcacctctgactgagcacgttgtacttcctccactctatgaggtcacgcctcacctcttcactaacagcgaagtcattgaatcagcttatcgtgccaagcagacacaaaaacctggtaaatttaagtctagtttcactggaaccaagaagaatcctgaacagagagtagcctatttcggagaggacattggaatgaacactcaccatgttacctggcacatggaattcccattctggtgggacgacaaatatagccatcatctggatcgcaaaggagagaacttcttctgggtacatcatcaacttaccgttcgctttgatgccgaacgtctctccaattacttagatcccgtcgaggaacttagctgggataaacccatcgtacaaggttttgctcctcacaccacttacaagtatggcggtcagttcccctctcgtccagataatgtagacttcgaggatgtggacggtgttgctcgcattcgagacttgcttattatcgagagccgaattcgcgatgctattgcccacggttatattgtcgataaggctggcaatcacattgacatcatgaatgagcgtggaatcgacgtccttggagatgttattgaatcatctttgtatagccctaacgtccagtactatggagctttgcacaatactgctcacattgtacttggtcgacagagtgatcctcatggaaagtacgctttaccccctggtgtactagaacactttgaaactgccacccgtgatcctagcttctttaggctgcataaatacatggataacatcttcaaggaacacaaggacagtctccctccctacacagtggaagaactaacatttgccggtgtaagtgtagacagcgtagcaattgaaggcgaactagagacctacttcgaggacttcgaatacaatcttattaacgccgtcgatgacactgaacagattgcagatgtagacatttctacttatgttcctcgtctcaaccacaaggactttaaaatcaaagttgatattagcaataacaagggcgaggaagttctagctaccgtgcgcattttcgcctggcctcacctcgacaacaatggtatcaagttcacctttgacgaaggccgttggaatgccattgaactggataagttctgggtcaagttgcctggtggaacacaccatatcgagcgtaaatgctcggaatctgctgttactgtgcctgatgcgcctagttttgcaacactctttgagaagaccaaagaagccttaggtggagcagactccggacttaaagatttcgagagtgccactggtattccaaatcgattcctcatccccaagggtaatgaacagggtctggagttcgaccttgttgttgccgtgactgatggcgcagccgacgcagcagtggatggcctccacgaaaacaccgaattcaatcattatggttcccatggcaagtaccctgacaatcgcccacatggctaccctctggatcgcagggttcccgatgaacgtgtattcgaagatcttcccaacttcggccacatccgagttaaggtcttcaatcatggcgagcatatccatcattaa

SEQ ID NO: 57 - protein, 680 aa, Litopenaeus vannamei
  MISC_FEATURE: Hemocyanin
WISAEFALLLALVAAAAAWPNFGFQSDAGGESDAQKQHDVNFLLHKIYGNIRDSDLKAKADSFDPAADLSHYSDGGEAVQKLVRDLKDDRLLQQRHWFSLFNPRQRHEALMLFDVLIHCKDWNTFVSNAAYFRQHMNEGEFVYALYVAVIHSPLTEHVVLPPLYEVTPHLFTNSEVIESAYRAKQTQKPGKFKSSFTGTKKNPEQRVAYFGEDIGMNTHHVTWHMEFPFWWDDKYSHHLDRKGENFFWVHHQLTVRFDAERLSNYLDPVEELSWDKPIVQGFAPHTTYKYGGQFPSRPDNVDFEDVDGVARIRDLLIIESRIRDAIAHGYIVDKAGNHIDIMNERGIDVLGDVIESSLYSPNVQYYGALHNTAHIVLGRQSDPHGKYALPPGVLEHFETATRDPSFFRLHKYMDNIFKEHKDSLPPYTVEELTFAGVSVDSVAIEGELETYFEDFEYNLINAVDDTEQIADVDISTYVPRLNHKDFKIKVDISNNKGEEVLATVRIFAWPHLDNNGIKFTFDEGRWNAIELDKFWVKLPGGTHHIERKCSESAVTVPDAPSFATLFEKTKEALGGADSGLKDFESATGIPNRFLIPKGNEQGLEFDLVVAVTDGAADAAVDGLHENTEFNHYGSHGKYPDNRPHGYPLDRRVPDERVFEDLPNFGHIRVKVFNHGEHIHH

SEQ ID NO: 58 - protein, 356 aa, Litopenaeus vannamei
  MISC_FEATURE: Arginine kinase
  MISC_FEATURE (1)..(18): IgE Epitope Sequence
  MISC_FEATURE (61)..(87): IgE Epitope Sequence
  MISC_FEATURE (78)..(96): IgE Epitope Sequence
  MISC_FEATURE (121)..(141): IgE Epitope Sequence
  MISC_FEATURE (160)..(189): IgE Epitope Sequence
  MISC_FEATURE (232)..(249): IgE Epitope Sequence
  MISC_FEATURE (319)..(342): IgE Epitope Sequence
MADAAVIEKLEAGFKKLEAATDCKSLLKKYLTKEVFDKLKDKKTSLGATLLDVIQSGVENLDSGVGIYAPDAEAYTLFAPLFDPIIEDYHVGFKQTDKHPNKDFGDVNSFVNVDPEGKFVISTRVRCGRSMQGYPFNPCLTESQYKEMEAKVSSTLSSLEGELKGTYYPLTGMSKEVQQKLIDDHFLFKEGDRFLQAANACRYWPAGRGIYHNDNKTFLVWVNEEDHLRIISMQMGGDLGQVFRRLTSAVNEIEKRIPFSHHDRLGFLTFCPTNLGTTVRASVHIKLPKLAANREKLEEVAGKYNLQVRGTRGEHTEAEGGIYDISNKRRMGLTEFQAVKEMQDGILELIKIEKEM

SEQ ID NO: 59 - protein, 177 aa, Litopenaeus vannamei; identical to SEQ ID NO: 4
  MISC_FEATURE: Myosin light chain mature polypeptide
  MISC_FEATURE (13)..(30): IgE Epitope Sequence
  MISC_FEATURE (22)..(48): IgE Epitope Sequence
  MISC_FEATURE (46)..(66): IgE Epitope Sequence
  MISC_FEATURE (58)..(96): IgE Epitope Sequence
  MISC_FEATURE (118)..(141): IgE Epitope Sequence

SEQ ID NO: 60 - protein, 193 aa, Litopenaeus vannamei; identical to SEQ ID NO: 6
  MISC_FEATURE: Sarcoplasmic calcium-binding protein
  MISC_FEATURE (10)..(33): IgE Eptiope Sequence
  MISC_FEATURE (49)..(72): IgE Epitope Sequence
  MISC_FEATURE (133)..(150): IgE Epitope Sequence

SEQ ID NO: 61 - protein, 284 aa, Litopenaeus vannamei; identical to SEQ ID NO: 16
  MISC_FEATURE: Tropomyosin
  MISC_FEATURE (1)..(39): IgE Epitope Sequence
  MISC_FEATURE (40)..(63): IgE Epitope Sequence
  MISC_FEATURE (61)..(81): IgE Epitope Sequence
  MISC_FEATURE (82)..(102): IgE Epitope Sequence
  MISC_FEATURE (115)..(153): IgE Epitope Sequence
  MISC_FEATURE (142)..(162): IgE Epitope Sequence
  MISC_FEATURE (157)..(186): IgE Epitope Sequence
  MISC_FEATURE (190)..(210): IgE Epitope Sequence
  MISC_FEATURE (247)..(284): IgE Epitope Sequence

SEQ ID NO: 62 - protein, 5 aa, Artificial Sequence
  Synthetic peptide
  MOD_RES (3)..(3): Xaa is Asp, Glu, Asn or Lys
  MOD_RES (4)..(4): Xaa is Asp or Glu
LEXXL

SEQ ID NO: 63 - protein, 5 aa, Artificial Sequence
  Synthetic peptide
  MOD_RES (3)..(3): Xaa is Asp, Glu, Asn or Lys
  MOD_RES (4)..(4): Xaa is Asp or Glu
LEXXN

SEQ ID NO: 64 - protein, 192 aa, Penaeus sp.
AYSWDNRVKYVVRYMYDIDDDGFLDKNDFECLAVRNTLIEGRGEFSAADYANNQKIMRNLWNEIAELADFNKDGEVTVDEFKMAVQKHCQGKKYSEFPGAFKVFIANQFKAIDVNGDGKVGLDEYRLDCITRSAFAEVKEIDDAYDKLTTEDDRKAGGLTLERYQDLYAQFISNPNESCSACFLFGPLKVVQ

SEQ ID NO: 65 - protein, 192 aa, Penaeus sp.
AYSWDNRVKYIVRYMYDIDNDGFLDKNDFECLAVRVTLIEGRGEFSPEGYAKNKEIMANLWNEIAELADFNKDGEVTVDEFKQAVQKNCKGKAFANFPNAFKVFIGNQFKTIDVDGDGMVGVDEYRLDCITRSAFADVKEIDDAYDKLCTEEDKKAGGINLARYQELYAQFISNEDEKNNACYLFGPLKEVQ

SEQ ID NO: 66 - protein, 193 aa, Procambarus clarkii
MAYSWDNRVKYVVRYMYDIDNNGFLDKNDFECLALRNTLIEGRGEFNEAAYANNQKIMSNLWNEIAELADFNKDGAVTIEQFKQAVQKNCSGKAFSAFPGAFKTFIANQFKTIDVNGDGLVGVDEYRLDCISRSAFANIKEIDDAYNKLATDEDKKAGGISLARYQELYAQFISNPDESANAVYLFGPLKEVQ

SEQ ID NO: 67 - protein, 176 aa, Mizuhopecten yessoensis
TDYLVSKWKIWYKSLDVNHDGIISIENVEESRNKFTDLHKLVGDKSTGVKVDMQKWWDTYIFLTPGAEISETQFVENLGNSFKKDKAFLATMTACFNMIFDVIDTDKDRSIDLNEFIYAFAAFGHENESVVRTAFALLKPDDDNTVPLRTVVDAWISFVTCEDASKTDVIKSAFES

SEQ ID NO: 68 - protein, 189 aa, Drosophila melanogaster
MAYSWDNRVDFVVRYMYDIDNNGFLDQNDFLCMAVRACVVEGKGDCSTARLDDYKKLMKNLWDEISAIADDDKDGKISNQEFKDAVKKTCVGKKYEEFPQAMRAFIESNFKLLDIDSDGIVGVKEYRYNCITRVAIDDITPIDKAFETLLNDEDRKRGGLSLDRYKELYGQFLGNTADNHPAVNLFGPL

SEQ ID NO: 69 - protein, 184 aa, Drosophila melanogaster
MSISDFRKKKLLFLFNVFFDVNQSGEIDVKDFELAIERVCQLRGWQKDTPKNKETYDLMMEIWTGLRSKADKDNDGQVSVDEWCNMWDAYAKDPSSVMDWQNAYMNFMFDLEDASHDGGIDVTEFTLVCSSYGLEKTECEEAFAKMSQGQSEVTREQFAALWKEYFAAEDVNAPGNYIFGKTSF